(19) United States
(12) Patent Application Publication
(10) Pub. No.: US 2022/0315888 A1
(43) Pub. Date: Oct. 6, 2022

(54) CULTURE METHOD FOR RETINAL ORGANOIDS

(71) Applicant: University of Newcastle Upon Tyne, Newcastle upon Tyne (GB)

(72) Inventors: Majlinda LAKO, Newcastle upon Tyne (GB); Darin ZERTI, Newcastle upon Tyne (GB); Birthe HILGEN, Newcastle upon Tyne (GB)

(21) Appl. No.: 17/642,199

(22) PCT Filed: Sep. 11, 2020
(86) PCT No.: PCT/GB2020/052207

§ 371 Date: Mar. 10, 2022

(30) Foreign Application Priority Data
Sep. 12, 2019 (GB) 1913196.0

(51) Int. Cl. C12N 5/0793; C12N 5/071; C12N 5/079; A61K 35/30

(52) U.S. Cl. CPC C12N 5/062; C12N 5/0697; C12N 5/0622; C12N 5/0621; A61K 35/30; C12N 2501/395; C12N 2501/42; C12N 2501/385; C12N 2501/105; C12N 2506/02; C12N 2513/00; C12N 2501/815; C12N 2506/45

(57) ABSTRACT

A method of producing a synthetic retina, including differentiating a stem cell culture in a culture medium and supplementing the culture with: (i) Triiodothyronine from about day 18 of cell differentiation; and (ii) retinoic acid for a first time period.

This invention relates to a culture method for producing a synthetic retina. Uses of the retina are also provided. The invention also provides defined media for use in the methods.

### BACKGROUND

Degenerative diseases of the retina represent one of the main causes of visual impairment and blindness, which culminate in the loss of photoreceptors and remain incurable [1]. Cell transplantation approaches for the replacement of lost or damaged photoreceptors have been investigated over the last decades in preclinical animal models of retinal degeneration [2]; thus, cell replacement has become a prerequisite on the path toward therapeutic transplantation. For this purpose human embryonic stem cells (hESCs) [3] and induced pluripotent stem cells (hiPSCs) [4] provide a suitable tool because both cell types can be expanded indefinitely and have the capacity to produce cone and rod precursors as well as more mature photoreceptor progeny in vitro [5]. The ability to generate retinal organoids from hESCs/hiPSCs under 3D culture conditions has been a great advance towards the generation of clinically relevant cell populations, that closely follow in vivo retinogenesis [6,7]. In the last seven years, intense work has been performed by several groups worldwide to improve the robustness and efficiency of differentiation protocols and to understand the factors and signalling pathways that are required to enhance retinal specification [8,9]. As such, developing methods to modulate cell composition and align differentiation states in vitro is critical for the path towards clinical transplantations.

Cell replacement therapy is a promising treatment for irreversible retinal cell death in diverse diseases, such as Stargardt's disease, Age-related macular degeneration (AMD) and Retinitis Pigmentosa (RP). The final impact in all these retinal dystrophies is the loss of all photoreceptors; hence, there is a pressing need for research into the replacement of photoreceptors. Seminal work has shown that a simple 3D culture system enables differentiation of human pluripotent stem cells to retinal organoids containing large numbers of photoreceptors developing alongside retinal neurons and Müller glia cells in a laminated structure that resembles the native retina. Despite these promising developments, current protocols show different efficiencies across pluripotent stem cells and result in retinal organoids with a mixture of photoreceptor cells at varying maturation states, along with non-photoreceptor cell types.

Accordingly, there remains a need for a method that is capable of producing sufficient numbers of photoreceptor precursors of the correct developmental stage to repair adult retina from hESC/hiPSC.

### BRIEF SUMMARY OF THE DISCLOSURE

The generation of retinal organoids derived from human pluripotent stem cells provides an in vitro model for disease modelling and replacement therapies. To date, the efficiency of protocols generating retinal organoids are variable, providing all retinal cell types including photoreceptor at different maturation stages. The inventors data shows that the addition of RA in combination with T3, LEVODOPA or DAPT, at specific time intervals, promotes cone and/or rod formation.

Specifically, the present invention is based on the surprising discovery that the addition of RA+T3 at specific stages of differentiation of retinal organoids, led to enhanced generation of rod and S-cone photoreceptors, which were able to form synaptic connections with the appropriate interneurons in the putative OPL. The inventors have shown that combined additions of RA+L-DOPA or RA+DAPT lead to selective enhancement either of S-cone photoreceptor or L/M-cone photoreceptor generation, respectively, at the expense of rod formation. Together the inventors data show that addition of specific reagents at selected differentiation time points can provide a useful strategy for the generation of retinal organoids enriched for specific photoreceptor subtype of interest.

In one aspect the invention provides a method of producing a synthetic retina, comprising differentiating a stem cell culture in a culture medium and supplementing the culture with:
(i) Triiodothyronine from about day 18 of cell differentiation; and
(ii) retinoic acid for a first time period from about day 90 to about day 120 of cell differentiation.

In one embodiment said culture is a three-dimensional stem cell culture, optionally wherein said three-dimensional stem cell culture is an embryoid body (EB) culture.

In one embodiment said culture medium is a neural cell culture medium.

In one embodiment said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one embodiment the method further comprises additionally supplementing the differentiation culture with one or more of 9-cis-retinal, 11-cis retinal, Levodopa and DAPT.

In one embodiment said additionally supplementing comprises supplementing said differentiation culture with retinoic acid and Levodopa for a second time period. In one embodiment said second time period is from about day 50 to about day 150, from about day 80 to about day 130, or preferably from about day 90 to about day 120 of cell differentiation culture. In one embodiment said additionally supplementing comprises supplementing the culture with retinoic acid and DAPT for a third time period. In one embodiment said third time period is from about day 10 to about day 150, from about day 20 to about day 130, or preferably from about day 30 to about day 120 of cell differentiation culture.

In one embodiment the method further comprises supplementing said culture with retinoic acid and Levodopa for a second time period and supplementing said culture with retinoic acid and DAPT for a third time period. In one embodiment said second time period is from about day 90 to about day 120 of cell culture and said third time period is from about day 30 to about day 120 of cell differentiation culture.

In one aspect the invention provides a method of producing a synthetic retina, comprising differentiating a stem cell culture in a culture medium and supplementing the culture with:
(i) Triiodothyronine from about day 18 of cell differentiation;
(ii) DAPT from about day 28 to about day 42 of cell differentiation; and
(iii) retinoic acid for a first time period from about day 30 to about day 120 of cell differentiation.

In one embodiment said culture is a three-dimensional stem cell culture, optionally wherein said three-dimensional stem cell culture is an embryoid body (EB) culture.

In one embodiment said culture medium is a neural cell culture medium.

In one embodiment said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one embodiment the method further comprising additionally supplementing the differentiation culture with one or more of 9-cis-retinal, 11-cis retinal, and Levodopa. In one embodiment said additionally supplementing comprises supplementing said differentiation culture with retinoic acid and Levodopa for a second time period. In one embodiment, wherein said second time period is from about day 50 to about day 150, from about day 80 to about day 130, or preferably from about day 90 to about day 120 of cell differentiation culture.

In one aspect the invention provides a method of promoting rod formation in a stem cell differentiation culture comprising supplementing the culture with:
(i) Triiodothyronine from about day 18 of cell differentiation; and
(ii) retinoic acid for a first time period from about day 90 to about day 120 of cell differentiation.

In one aspect the invention provides a method of promoting S, L and/or M cone formation in a stem cell differentiation culture comprising supplementing the culture with:
(i) Triiodothyronine from about day 18 of cell differentiation;
(ii) DAPT from about day 28 to about day 42 of cell differentiation; and
(iii) retinoic acid for a first time period from about day 30 to about day 120 of cell differentiation.

In accordance with the present inventions there is also provided a method of producing a synthetic retina, comprising differentiating a stem cell culture in a culture medium and supplementing the culture with retinoic acid and Triiodothyronine for a first time period from about day 50 to about day 150 of cell differentiation.

In one embodiment said culture is a three-dimensional stem cell culture, optionally wherein said three-dimensional stem cell culture is an embryoid body (EB) culture.

In one embodiment said culture medium is a neural cell culture medium.

In one embodiment said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one embodiment said first time period is from about day 80 to about day 130, preferably from about day 90 to about day 120 of cell differentiation culture.

In one embodiment said method further comprises additionally supplementing the differentiation culture with one or more of 9-cis-retinal, 11-cis retinal, Levodopa and DAPT.

In one embodiment said additionally supplementing comprises supplementing said differentiation culture with retinoic acid and Levodopa for a second time period. Preferably, wherein said second time period is from about day 50 to about day 150, from about day 80 to about day 130, or preferably from about day 90 to about day 120 of cell differentiation culture.

In one embodiment said additionally supplementing comprises supplementing the culture with retinoic acid and DAPT for a third time period. Preferably, said third time period is from about day 10 to about day 150, from about day 20 to about day 130, or preferably from about day 30 to about day 120 of cell differentiation culture.

In one embodiment said method further comprises supplementing said culture with retinoic acid and Levodopa for a second time period and supplementing said culture with retinoic acid and DAPT for a third time period. Preferably, wherein said second time period is from about day 90 to about day 120 of cell culture and said third time period is from about day 30 to about day 120 of cell differentiation culture.

In one embodiment said first time period is from about day 90 to about day 120 of cell differentiation culture.

In a further aspect the invention provides a method of promoting rod formation and/or S cone formation in a stem cell differentiation culture comprising supplementing said culture with retinoic acid and Triiodothyronine for a period from about day 50 to about day 150 of cell culture. Preferably, said period is from about day 80 to about day 130, preferably from about day 90 to about day 120 of cell culture.

In a further aspect the invention provides a method of promoting S cone formation in a stem cell differentiation culture comprising supplementing said culture with retinoic acid and Levodopa for a period from about day 50 to about day 150 of culture. Preferably, wherein said period is from about day 80 to about day 130, preferably from about day 90 to about day 120 of cell culture

In a further aspect the invention provides a method of promoting L cone and/or M cone formation in a stem cell differentiation culture comprising supplementing said culture with retinoic acid and DAPT for a period from about day 10 to about day 150 of culture. Preferably, wherein said period is from about day 20 to about day 130, or preferably from about day 30 to about day 120 of cell culture.

In a further aspect the invention provides a method of producing a synthetic retina, comprising differentiating a stem cell culture in a cell culture medium and supplementing the culture with:
(i) retinoic acid for a first time period from about day 10 of cell culture to about at least about 10 days prior to the end of differentiation, or until the end of differentiation;
(ii) Levodopa and/or Triiodothyronine for a second time period from about day 50 of cell culture to about at least about 10 days prior to the end of differentiation; and
(iii) DAPT for a third time period from about day 10 of cell culture to about at least about 10 days prior to the end of differentiation.

In one embodiment said first time period is from about day 25, day 30, day 35 or day 40 to about at least about 10 days prior to the end of differentiation. Optionally or alternatively said the first time period is to about at least 10, at least 20, at least 20, at least 30, at least 40 days prior to the end of differentiation.

In one embodiment said second time period is from about day 50, day 60, day 70, day 80, day 90, or day 100 to about at least about 10 days prior to the end of differentiation. Optionally or alternatively wherein the second time period is to about at least 10, at least 20, at least 20, at least 30, at least 40 days prior to the end of differentiation.

In one embodiment said third time period is from is from about day 25, day 30, day 35 or day 40 to about at least about 10 days prior to the end of differentiation. Optionally or alternatively, wherein the third time period is to about at least 10, at least 20, at least 20, at least 30, at least 40 days prior to the end of differentiation.

In one embodiment said method further comprises supplementing the culture with Levodopa and Triiodothyronine for the second time period.

In one embodiment said end of culture is at about day 300, day 250, day 200, or day 150 of cell differentiation. Preferably, wherein the end of differentiation is at about day 150. Still more preferably wherein the first time period if from about day 30 to about day 120 of cell differentiation.

In one embodiment said second time period is from about day 90 to about day 120 of cell differentiation. Preferably, wherein said third time period is from about day 30 to about day 120 of cell differentiation.

In one embodiment said culture is a three-dimensional stem cell culture, optionally wherein said three-dimensional stem cell culture is an embryoid body (EB) culture.

In one embodiment said culture medium is a neural cell culture medium.

In one embodiment said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one embodiment said stem cell culture consists of human induced pluripotent stem cells (hiPSC) or human embryonic stem cells (hESC).

In one embodiment said the method further comprises a step of isolating said synthetic retina from said cell culture medium. Optionally, wherein the method further comprises a step of extracting at least one photoreceptor from said synthetic retina.

In a further aspect the invention provides a synthetic retina obtainable by the method of the invention.

In a further aspect the invention provides a photoreceptor obtainable by the method of the invention.

In a further aspect the invention provides a pharmaceutical composition comprising said synthetic retina according to the invention or said photoreceptor according to the invention.

In a further aspect the invention provides the synthetic retina according to the invention, a photoreceptor according to the invention or the pharmaceutical composition according to the invention for use in the treatment of retinal disease or ocular injury.

In a further aspect the invention provides a method of treating a retinal disease or an ocular injury comprising implanting the synthetic retina according to the invention or the photoreceptor according to the invention into the eye of a mammalian subject in need thereof, or administering the pharmaceutical composition according to the invention into the eye into the eye of a mammalian subject in need thereof.

In one embodiment the retinal disease is a retinal degenerative disease.

In one embodiment the retinal degenerative disease is selected from the group consisting of Retinitis Pigmentosa, age-related macular degeneration, Bardet-Biedel syndrome, Bassen-Kornzweig syndrome, Best disease, choroideremia, gyrate atrophy, Leber congenital amaurosis, Refsun syndrome, Stargardt disease or Usher syndrome.

In a further aspect the invention provides the synthetic retina according to the invention or the photoreceptor according to the invention for use as a tissue graft.

In one embodiment said stem cell culture consists of human induced pluripotent stem cells (hiPSC) obtained from a subject to be treated.

In a further aspect the invention provides use of the synthetic retina according to the invention or the photoreceptor of the invention in an in vitro model for retinal or neurological disease.

In a further aspect the invention provides use of a synthetic retina according to the invention or a photoreceptor of the invention in a screen to identify compounds useful in the treatment of retinal or neurological disease.

### BRIEF DESCRIPTION OF THE DRAWINGS

Embodiments of the invention are further described hereinafter with reference to the accompanying drawings, in which:

FIG. 1: Diagram of the experimental design, showing the addition of Retinoic Acid (RA), 9-cis-retinal, 11-cis-retinal, Levodopa (L-DOPA), Triiodothyronine (T3) and DAPT individually or in combination with each other as well as the emergence of retinal cell types at different time points (day 30-60, day 60-90, day 90-120 and day 30-120) during the differentiation of retinal organoids. Please note that DAPT was added from day 30-42 (for day 30-60 combined addition with RA from day 30-60), day 60-72 (for the combined addition with RA from day 60-90), day 90-102 (for the combined addition with RA from day 90-120) and day 30-42 (for the combined addition with RA from day 30-120).

FIG. 2: Characterization of Rhodopsin expression in retinal organoids derived from hESCs at day 150 of differentiation. A) Gene expression analysis of Rhodopsin at all time points during differentiation revealed a significant increase in RA+T3 day 90-120 condition compared to vehicle control at day 90-120. B) Representative examples of Rhodopsin immunoreactivity (red and arrow) in all conditions (a-g) from day 90-120 stage specific additions, showing the highest number of Rhodopsin+ cells in RA+T3 condition (g). Higher magnification demonstrated the typical morphology of rod photoreceptors (g′). Double staining with Rhodopsin (magenta) and Synaptophysin (red) indicated the possible formation of synapses in the developing OPL (h). CRX (green) represents the endogenous GFP expression and nuclei are counterstained with Hoechst (blue). Scale bars, 50 μm (B a-h) 10 μm (B g′). C) Immunohistochemistry quantification revealed a reduction of Rhodopsin+ cells in RA, 11-cis-retinal, RA+L-DOPA and RA+DAPT condition and a significant increase in RA+T3 condition compared to vehicle control. Data is shown as mean±SEM (n=5) and statistical significant differences were considered at *p<0.05, **p<0.01, ***p<0.001, ****p<0.0001. Abbreviations: L-DOPA, Levodopa; OPL, outer plexiform layer; RA, Retinoic Acid; RHO, Rhodopsin; T3, Triiodothyronine.

FIG. 3: Characterization of OPN1SW (S cones) expression in retinal organoids derived from hESCs at day 150 of differentiation. A) qRT-PCR analysis of OPN1SW at all time points during differentiation showed an increase in RA+T3 condition compared to vehicle control (p<0.05) at day 90-120. B) Expression of OPN1SW (magenta and arrow) was found in all conditions at time point day 90-120 (a-g). The highest number of OPN1SW+ cells was observed in RA+T3 and RA+L-DOPA conditions (g, d) highlighting cells in the higher magnification image (g′). Double staining with OPN1SW (magenta) and Bassoon (red; h) or Ribeye (red; i), receptively, indicated putative ribbon synapse formation in the developing OPL (arrowheads). CRX (green) represents the endogenous GFP expression and nuclei are counterstained with Hoechst (blue). Scale bars, 50 μm (B a-i) 10 μm (B g′). C) IHC quantification analysis showed a significant increase of OPN1SW+ cells in RA+L-DOPA and RA+T3 condition compared to vehicle control. Data is shown as mean±SEM (n=5) and statistical significant differences were considered at **p<0.01, ***p<0.001. Abbreviations: L-DOPA, Levodopa; OPL, outer plexiform layer; RA, Retinoic Acid; T3, Triiodothyronine.

FIG. 4: Characterization of OPN1MW/LW (L/M cones) expression in retinal organoids derived from hESCs at day 150 of differentiation. A) Gene expression analysis of OPN1MW

(left) and OPN1LW (right) at all time points during differentiation revealed a significant increase in the expression of both genes in RA+DAPT condition compared to vehicle control at day 30-120. B) Representative examples of OPN1MW/LW expression (red and arrow) in all conditions at time point day 30-120 (a-g), indicating the highest number OPN1MW/LW+ cells in RA+DAPT condition (g). Higher magnification demonstrated the morphology of L/M cones. (g′) Ribbon synapse formation was demonstrated by double staining of OPN1MW/LW (magenta) with Bassoon (red; h) or Ribeye (red; i), receptively. CRX (green) represents the endogenous GFP expression and nuclei are counterstained with Hoechst (blue). Scale bars, 50 μm (B a-i) 10 μm (B g′). C) Quantification of OPN1MW/LW+ cells revealed a significant increase of L/M cones in RA+DAPT condition compared to vehicle control. Data is shown as mean±SEM (n=5) and statistical significant differences were considered at *p<0.05. Abbreviations: L-DOPA, Levodopa; RA, Retinoic Acid; T3, Triiodothyronine.

FIG. 5: Individual channels images of Rhodopsin immunoreactivity (red), CRX (green) represents the endogenous GFP expression and nuclei are counterstained with Hoechst (blue) in all conditions (A-H) from day 90-120 stage specific additions, showing the highest number of Rhodopsin+ cells in RA+T3 condition (F, F′ and F″). Higher magnification showed the nuclei in blue (G), the endogenous GFP expression in the apical layer (G′) and the typical morphology of rod photoreceptors (G″). Individual channel of the double staining with Rhodopsin (magenta) and Synaptophysin (red) indicated the possible formation of synapses in the developing OPL (I and I″); CRX (green) represents the endogenous GFP expression (I′). Scale bars 50 μm (A-I″).

FIG. 6: Individual channels images of OPN1SW immunoreactivity (magenta), CRX (green) represents the endogenous GFP expression and nuclei are counterstained with Hoechst (blue) in all conditions (A-H) from day 90-120 stage specific additions, showing the highest number of OPN1SW+ cells in RA+T3 condition (G, G′ and G″). Higher magnification showed the nuclei in blue (H), the endogenous GFP expression in the apical layer (H′) and the typical morphology of S cone photoreceptors (H″). Individual channels of the double staining with OPN1SW (magenta) and Bassoon (red) (I and I″); OPN1SW (magenta) and Ribeye (red) (J and J″) indicated, respectively the possible formation of synapses in the developing OPL; CRX (green) represents the endogenous GFP expression (I′ and J′). Scale bars 50 μm (A, A′, A″, B, B′, B″, C, C′, C″, D, D′, D″, E, E′, E″, F, F′, F″, G, G′, G″, I, I′, I″, J, J′ and J″) and 10 μm (H, H′ and H″).

FIG. 7: Diagram showing the percentage of photoreceptors within the retinal organoids divided into S cones (blue), L/M cones (green) and rods (red) in all conditions. Data are presented as average of two time points: day 90-120 and day 30-120.

FIG. 8: Individual channels images of OPN1MW/LW immunoreactivity, CRX represents the endogenous GFP expression and nuclei are counterstained with Hoechst in all conditions (A-H) from day 30-120 stage specific additions, showing the highest number of OPN1MW/LW+ cells in RA+DAPT condition (G, G′ and G″). Higher magnification showed the nuclei in blue (H), the endogenous GFP expression in the apical layer (H′) and the typical morphology of L/M cone photoreceptors (H″). Individual channels of the double staining with OPN1MW/LW and Bassoon (red) (I and I″); OPN1MW/LW and Ribeye (J and J″) indicated the possible formation of synapses in the developing OPL. CRX represents the endogenous GFP expression (I′ and J′). Scale bars 50 μm (A, A′, A″, B, B′, B″, C, C′, C″, D, D′, D″, E, E′, E″, F, F′, F″, G, G′, G″, I, I′, I″, J, J′ and J″) and 10 μm (H, H′ and H″).

FIG. 9: Characterisation of retinal organoid lamination in RA+DAPT condition at day 150 of differentiation. A) Representative brightfield images of retinal organoids at day 150, showing bright phase neuroepithelium on the apical side of organoids. B) CRX (endogenous GFP expression; green) and Recoverin (red) expression was found at the apical edge of organoids, forming a putative ONL. C) Some NRL+ cells (red) were found in the photoreceptor layer at the apical edge of retinal organoids. D) Expression of Arrestin3 (red) was observed above photoreceptor nuclei in the developing photoreceptor inner/outer segments. E) RXRγ+ cells (red) were seen throughout the retinal organoid. F) Ganglion cells detected by HuC/D (red) were located in the middle of retinal organoids, forming a putative GCL. CRX (green) represents the endogenous GFP expression and nuclei are counterstained with Hoechst (blue). Abbreviations: ONL, outer nuclear layer; GCL, ganglion cell layer. Scale bars, 200 pixel (A) and 50 μm (B-F). Abbreviations: RA, Retinoic Acid.

FIG. 10: Brightfield representative examples of retinal organoids at day 150 of differentiation for RA+T3 (A-D) and RA+DAPT (E-H) conditions. In both conditions, neural retinal and RPE are visible. Scale bar, 200 pixel. Abbreviations: RA, Retinoic Acid; T3, Triiodothyronine.[ML1]

FIG. 11 provides a schematic summary of our results showing that the addition of RA+T3 (day 90-120) led to enhanced generation of rod and S-cone photoreceptors while combined addition of RA+L-DOPA, at the same time window, promoted only the S-cone formation. The supplementation of RA+DAPT (day 30-120) resulted in an increased L/M-cone photoreceptor generation.

FIG. 12: shows the amino acid sequence of human IGF-1 (as shown at http://www.ncbi.nlm.nih.gov/gene/3479#reference-sequences IGF-1 RefSeqGene: http://www.ncbi.nlm.nih.gov/nuccore/NG_011713.1?from=5001&to=89734&report=genbank).

FIG. 13: Individual channels images of Rhodopsin immunoreactivity, and nuclei are counterstained with Hoechst in all conditions from day 90-120 stage specific additions. A graph summarising the percentage of Rhodopsin positive cells in all conditions is shown on the bottom right hand corner.

FIG. 14 shows Opsin Blue staining in HiPSCs. Nuclei are counterstained with Hoechst in all conditions. A graph summarising the percentage of Opsin Blue positive cells in all conditions is shown on the bottom right hand corner.

FIG. 15 shows Opsin red/green in hiPSC and hESCs. Nuclei are counterstained with Hoechst in all conditions. A graph summarising the percentage of red/green opsin positive cells in all conditions is shown on the bottom right hand corner.

### DETAILED DESCRIPTION

Under basal media conditions, hESC and hiPSC are capable of exhibiting neural, eye field, retinal and photoreceptor gene expression over time and can produce various retinal phenotypes in the absence of inductive cues, suggestive of an intrinsic ability that can be exploited to optimise the differentiation process. (Meyer, J. S., Howden, S. E., Wallace, K. A., Verhoeven, A. D., Wright, L. S. et al. Optic vesicle-like structures derived from human pluripotent stem cells facilitate a customized approach to retinal disease treatment. Stem Cells. 29(8), 1206-18 (2011); Mellough, C. B., Sernagor, E., Moreno-Gimeno, I., Steel, D. H., Lako, M. Efficient stagespecific differentiation of human pluripotent stem cells toward retinal photoreceptor cells. Stem Cells. 30(4), 673-86 (2012)).

The inventors have now surprisingly shown that the stage-specific addition of Retinoic Acid (RA), 9-cis-retinal, 11-cis-retinal, Levodopa (L-DOPA), Triiodothyronine (T3) and gamma-secretase inhibitor (DAPT) can control the generation of cone and rod photoreceptors. The results indicate that addition of RA+T3 during day 90-120 of differentiation enhanced the generation of rod and S-cone photoreceptor formation, whilst the addition of RA+DAPT during day 30-120 of differentiation leads to enhanced generation of L/M-cones at the expense of rods. Levodopa when added together with RA during day 90-120 of differentiation promotes the emergence of S-cones at the expense of rod photoreceptors. Collectively these data represent an advance in the ability to direct generation of rod and cone photoreceptors in vitro.

The inventors have now surprisingly identified that the addition of retinoic acid (RA) and Triiodothyronine (T3) to hESC/hiPSC cultures for a defined period of differentiation enhances the generation of rod and S-cone photoreceptor generation.

The inventors have additionally surprisingly identified that the addition of retinoic acid (RA) and gama-secretase inhibitor (DAPT) to hESC/hiPSC cultures for a defined period of differentiation enhances the generation of UM-cones at the expense of rod photoreceptors.

The inventors have additionally surprisingly identified that the addition of retinoic acid (RA) and Levodopa to hESC/hiPSC cultures for a defined period of differentiation enhances the generation of S-cones at the expense of rod photoreceptors.

Singularly and collectively, these findings can be used in the directed differentiation of pluripotent or multipotent stem cells, including human embryonic stem cells (hESC), somatic stem cells, and induced human pluripotent stem cells (hiPSC), towards the generation of rod and cone photoreceptors in vitro.

### Cells and Culture Conditions

As used herein, the terms “culture” and “cell culture” are used interchangeably refer to the process whereby cells, preferably stem cells, are grown under controlled conditions, preferably in vitro.

As used herein the terms “differentiate” and “differentiation” relate to the process by which an unspecialised or relatively less specialised cell becomes relatively more specialised. In the context of cell ontogeny, the adjective “differentiated” is a relative term. Hence, a “differentiated cell” is a cell that has progressed further down a certain developmental pathway than the cell it is being compared with. The term “differentiation” as used with respect to cells in a differentiating cell culture refers to the process by which cells differentiate from one cell type (e.g., a multipotent, totipotent or pluripotent differentiable cell) to another cell type such as a target differentiated cell (e.g. a photoreceptor). The term “cell differentiation” in reference to a pathway refers to a process by which a less specialized cell (i.e. stem cell) develops or matures or differentiates to possess a more distinct form and/or function into a more specialized cell or differentiated cell, (i.e. a photoreceptor).

As used here in the term “stem cells” refers to pluripotent cells characterised by indefinite self-renewal ability and the capacity to give rise to any cell type in the adult. The term includes human embryonic stem cells (hESC) and human induced pluripotent stem cells (hiPSCs). hESC are derived from spare in vitro fertilised embryos after parental consent and have been widely used in the last decade as a generic tool to understand maintenance of pluripotency, human embryonic development and congenital disease. Destruction of human embryos for research purposes is surrounded by a number of ethical issues, prohibiting hESC derivation and application in several countries. However the main issue related to their application is the evidence that their differentiated progeny express human leukocyte antigens (HLAs) that will probably result in graft rejection and could be bypassed only by creation of HLA-typed hESC banks, from which a best match can be selected. Human iPSC bypass both of these issues as they are generated by reprogramming somatic cells back to the pluripotent state akin to embryonic stem cells. As such, these cells share all the characteristics of hESC including the ability to proliferate indefinitely and differentiate into many cell types, but also represent a source of autologous stem cells for cell replacement therapies given that they are derived from the patient themselves. Such patient derived cells present a unique opportunity to create in vitro disease models which can be exploited to understand disease pathology and drug discovery. This becomes extremely important for degenerative diseases such as those affecting the retina, where availability of patient specific cells (i.e. photoreceptors and RPE) becomes a possibility only after invasive surgery or death. New tools developed in the gene therapy field including improved and safer viral vectors as well as the possibility of correcting endogenous mutations through the application of site specific restriction endonucleases (such as Zinc Finger Nucleases, known as ZFN or Transcription Activator-Like Effector Nucleases also known as TALEN), also mean that functional patient specific cells for transplantation can be produced from hiPSC.

As used herein the term “medium” and “media” are used interchangeably.

As used herein “neural cell culture medium” is used to refer to a culture media containing the minimum essential elements necessary to maintain the growth of a neural cell. Such neural cell culture media typically comprise roughly fifty chemical entities at known concentrations in water. The chemical components of the culture media fall into five broad categories: amino acids, vitamins, inorganic salts, trace elements, and a miscellaneous category that defies neat categorization. In addition, neural cell culture medium preferably comprises glucose, most preferably D-glucose.

Defined culture media comprising minimum essential elements necessary to maintain the growth of neural cells are well known in the art and include, by way of example only Minimum Essential Medium Dulbecco, F12 (HAM), RPMI 1640, advanced RPMI, Dulbecco's Modified Eagle Medium (DMEM—without serum), Knockout DMEM, Knockout DMEM:F12, DMEM (high glucose), Neurobasal, Neurobasal A, Glasgow Modification Eagle Medium (GMEM), Leibovitz L-15 Medium, McCoy's 5A Medium and Waymouth's medium.

In one instance the neural cell culture medium comprises an IGF-1 receptor agonist. In another instance the neural cell culture medium comprises: a) L-glutamine; b) B27 supplement; and c) an IGF-1 receptor agonist. In a further instance the neural cell culture medium comprises: a) L-glutamine; b) B27 supplement; c) N2 supplement; and d) an IGF-1 receptor agonist.

As used herein “827 supplement” refers to a neural cell culture supplement, such as B-27® serum-free supplement (Life Technologies). Preferably, the supplement comprises Probably T3, insulin and vitamin A.

As used herein “N-2 supplement” refers to a supplement for the growth and expression of post-mitotic neurons and tumor cells of neuronal phenotype, for example a Bottenstein's N-1 or N-2 supplement, most preferably Bottenstein's N-2 supplement (Life Technologies). Preferably, the supplement comprises Human Transferrin and Insulin, more preferably the supplement comprises Human Transferrin, Insulin, Progesterone, Putrescine and Selenite.

In one instance, a preferred medium for use in the neural cell culture medium of the present invention comprises the commercially available media DMEM-Hams F-12 (Life Technologies).

The neural cell culture medium is supplemented with an (insulin-like growth factor) IGF-1 receptor agonist. As used herein, the term “IGF-1 receptor agonist” refers to any compound, for example a peptide, which is capable of acting at insulin-like growth factor receptor as an agonist, i.e. acts as a ligand that elicits insulin-like growth factor receptor activity. Preferably the agonist is Insulin-like growth factor 1 (IGF-1). More preferably, the IGF-1 is human IGF-1, or a homologue or analogue thereof.

IGF-I is a single-chain, 70-amino-acid protein with high homology to proinsulin, the sequence of which is shown in FIG. 12 (UniProtKB P00750, P05019-2, Isoform 2, also known as IGF-1A). Unlike the other members of the insulin superfamily, the C region of -IGF's is not proteolytically removed after translation. The solution NMR structures of IGF-I (Cooke et al., Biochemistry, 30: 5484-5491 (1991); Hua et al., J. Mol. Biol., 259: 297-313 (1996)), mini-IGF-I (an engineered variant lacking the C-chain; DeWolf et al., Protein Science, 5: 2193-2202 (1996)), and IGF-II (Terasawa et al., EMBO J. 13: 5590-5597 (1994); Torres et al., J. Mol. Biol. 248: 385-401 (1995)) have been reported. It is generally accepted that distinct epitopes on IGF-I are used to bind receptor and binding proteins. It has been demonstrated in animal models that receptor-inactive IGF mutants are able to displace endogenous IGF-I from binding proteins and thereby generate a net IGF-I effect in vivo (Loddick et al., Proc. Natl. Acad. Sci. USA, 95: 1894-1898 (1998); Lowman et al., Biochemistry, 37: 8870-8878 (1998); U.S. Pat. Nos. 6,121,416 and 6,251,865).

Preferably the IGF-1 is at least 60%, 65%, 70%, 75%, 80%, 85%, 90%, 91%, 92%, 93%, 94%, 95%, 96%, 97%, 98%, 99% or 100% identity to the entire length of the polypeptide sequence shown in SEQ ID NO:1.

As used herein, the terms “homology” and “identity” are used interchangeably. Calculations of sequence homology or identity between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

Many mutagenesis studies have addressed the characterization of the IGFBP-binding epitope on IGF-I (Bagley et al., Biochem. J., 259: 665-671 (1989); Baxter et al., J. Biol. Chem., 267: 60-65 (1992); Bayne et al., J. Biol. Chem., 263: 6233-6239 (1988); Clemmons et al., J. Biol. Chem., 265: 12210-12216 (1990); Clemmons et al., Endocrinology, 131: 890-895 (1992); Oh et al., supra.

Variants of IGF-1 have been disclosed. WO 96/33216 describes a truncated variant having residues 1-69 of authentic IGF-I. EP742228 discloses two-chain IGF-I superagonists that are derivatives of the naturally occurring single-chain IGF-I having an abbreviated C domain. Cascieri et al., Biochemistry, 27: 3229-3233 (1988) discloses mutants of IGF-I,] Cascieri et al., J. Biol. Chem., 264: 2199-2202 (1989) discloses three IGF-I analogs in Sliecker et al., Adv. Experimental Med. Biol., 343: 25-32 (1994)) describes the binding affinity of various IGF and insulin variants to IGFBPs, IGF receptor, and insulin receptor. The IGF-1 agonist may be an IGF-1 variant.

The neural cell culture medium may be a serum free culture media, i.e. a medium that contains no serum (e.g., fetal bovine serum (FBS), horse serum, goat serum, etc.). In some instances, the neural cell culture medium is supplemented with a serum-free eukaryotic cell culture medium supplement, for example KnockOut™ Serum Replacement media supplement, Essential 6 Medium (both Life Technologies), and TeSR-E6 (Stem Cell Technologies).

The neural cell culture medium is supplemented with an amino acid, such as a Non-Essential Amino Acid media supplement (NEAA, Life Technologies).

The neural cell culture medium may be further supplemented with antibiotics, albumin, amino acids, or other components known in the art for the culture of cells.

In one instance the neural cell culture medium comprises: a) Dulbecco's Modified Eagle Medium (DMEM), Nutrient Mixture F-12 (F-12), or a combination thereof; b) knockout serum replacement (KOSR); c) L-glutamine; d) Non essential amino acid (NEAA); e) B27 supplement; and f) an IGF-1 receptor agonist.

In one instance the neural cell culture medium comprises: a) Dulbecco's modified Eagle Medium (DMEM), Nutrient Mixture F-12 (F-12), or a combination thereof; b) L-glutamine; c) Non essential amino acid (NEAA); d) B27 supplement; e) N2 supplement; and f) an IGF-1 receptor agonist.

In one instance the first neural cell culture medium comprises: a) Dulbecco's Modified Eagle Medium (DMEM), Nutrient Mixture F-12 (F-12), or a combination thereof; b) knockout serum replacement (KOSR); c) L-glutamine; d) Non essential amino acid (NEAA); e) B27 supplement; and f) an IGF-1 receptor agonist and the first neural cell culture medium consists of: a) Dulbecco's modified Eagle Medium (DMEM), Nutrient Mixture F-12 (F-12), or a combination thereof; b) L-glutamine; c) Non essential amino acid (NEAA); d) B27 supplement; e) N2 supplement; and f) an IGF-1 receptor agonist.

Such neural cell culture media are known in the art (see for example Dorgau et al, Biomaterials. 2019 April; 199:63-75; Collin et al Stem Cells. 2019 May; 37(5):609-622; Collin et al Stem Cells. 2019 May; 37(5):593-598; Dorgau et al, Cell Death Dis. 2018 May 23; 9(6):615; and Felemban et al, Acta Biomater. 2018 Jul. 1; 74:207-221).

DAPT is an inhibitor of the γ-secretase complex. Notch is a key target of γ-secretase, therefore DAPT indirectly inhibits the Notch pathway.

Levodopa—L-DOPA (also known as L-3,4-dihydroxyphenylalanin) e, is an amino acid that is made and used as part of the normal biology of humans, as well as some animals and plants.

Triiodothyronine, also known as T3, is a thyroid hormone.

Retinoic acid (used simplified here for all-trans-retinoic acid) is a metabolite of vitamin A₁ (all-trans-retinol) that mediates the functions of vitamin A₁ required for growth and development.

In one aspect the invention provides a method of producing a synthetic retina, comprising differentiating a stem cell culture in a culture medium and supplementing the culture with:
(i) Triiodothyronine from about day 18 of cell differentiation; and
(ii) retinoic acid for a first time period from about day 90 to about day 120 of cell differentiation.

The supplementation with retinoic acid and Triiodothyronine may be simultaneous, concomitant, sequential, or adjunctive.

In one instance Triiodothyronine is added until the end of culture. The end of culture may be at about day 300, day 250, day 200, or day 150 of cell differentiation.

In one instance said culture is a three-dimensional stem cell culture, optionally wherein said three-dimensional stem cell culture is an embryoid body (EB) culture.

In one instance said culture medium is a neural cell culture medium.

In one instance said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one instance the method further comprises additionally supplementing the differentiation culture with one or more of 9-cis-retinal, 11-cis retinal, Levodopa and DAPT. In one embodiment said additionally supplementing comprises supplementing said differentiation culture with retinoic acid and Levodopa for a second time period. In one instance said second time period is from about day 50 to about day 150, from about day 80 to about day 130, or preferably from about day 90 to about day 120 of cell differentiation culture. In one instance said additionally supplementing comprises supplementing the culture with retinoic acid and DAPT for a third time period. In one instance said third time period is from about day 10 to about day 150, from about day 20 to about day 130, or preferably from about day 30 to about day 120 of cell differentiation culture.

In one instance the method further comprises supplementing said culture with retinoic acid and Levodopa for a second time period and supplementing said culture with retinoic acid and DAPT for a third time period. In one instance said second time period is from about day 90 to about day 120 of cell culture and said third time period is from about day 30 to about day 120 of cell differentiation culture.

In one instance the culture is supplemented with about 5, 10, 15, 20, 25, 30, 34, 40, 45, 50, 55 or 60 ng/ml of Triiodothyronine per day.

In one instance the culture is supplemented with about 0.1, 0.2, 0.3, 0.4, 0.5, 0.6, 0.7, 0.08, 0.9, 1.0, 1.1, 1.2, 1.3, 1.4, 1.5, 1.6, 1.7, 1.8, 1.9, or 2.0 μM of RA per day.

In one instance the culture is supplemented with about 10-about 55, preferably about 15-about 45, more preferably about 20-about 40 ng/ml Triiodothyronine per day.

In one instance the culture is supplemented with about 0.1-1.8, about 0.2-about 1.5, about 0.3-about 1.5, about 0.4-about 1.3, or preferably about 0.5-about 1.0 μM of RA per day.

In one instance the culture is supplemented with about 20 to about 40 ng/ml Triiodothyronine per day and 0.5 to about 1.0 μM of RA per day.

In one aspect the invention provides a method of producing a synthetic retina, comprising differentiating a stem cell culture in a culture medium and supplementing the culture with:
(i) Triiodothyronine from about day 18 of cell differentiation;
(ii) DAPT from about day 28 to about day 42 of cell differentiation; and
(iii) retinoic acid for a first time period from about day 30 to about day 120 of cell differentiation.

The supplementation with retinoic acid and Triiodothyronine may be simultaneous, concomitant, sequential, or adjunctive.

In one instance Triiodothyronine is added until the end of culture. The end of culture may be at about day 300, day 250, day 200, or day 150 of cell differentiation.

In one instance said culture is a three-dimensional stem cell culture, optionally wherein said three-dimensional stem cell culture is an embryoid body (EB) culture.

In one instance said culture medium is a neural cell culture medium.

In one instance said culture media is supplemented with an IGF-1 receptor agonist, optionally wherein said IGF-1 receptor agonist is selected from the group consisting of:
i) a human IGF-1;
ii) a homologue of human IGF-1; or
iii) an analogue of human IGF-1.

In one instance the method further comprising additionally supplementing the differentiation culture with one or more of 9-cis-retinal, 11-cis retinal, and Levodopa. In one instance said additionally supplementing comprises supplementing said differentiation culture with retinoic acid and Levodopa for a second time period. In one instance, wherein said second time period is from about day 50 to about day 150, from about day 80 to about day 130, or preferably from about day 90 to about day 120 of cell differentiation culture.

In one instance the culture is supplemented with about 5, 10, 15, 20, 25, 30, 34, 40, 45, 50, 55 or 60 ng/ml of Triiodothyronine per day.

In one instance the culture is supplemented with about 0.1, 0.2, 0.3, 0.4, 0.5, 0.6, 0.7, 0.08, 0.9, 1.0, 1.1, 1.2, 1.3, 1.4, 1.5, 1.6, 1.7, 1.8, 1.9, or 2.0 μM of RA per day.

In one instance the culture is supplemented with about 1, 2, 3, 4, 5, 6, 7, 8, 9, 10, 11, 12, 13, 14, 15, 16, 17, 18, 19, or 20 um DATP per day.

In one instance the culture is supplemented with about 10-about 55, preferably about 15-about 45, more preferably about 20-about 40 ng/ml Triiodothyronine per day.

In one instance the culture is supplemented with about 0.1-1.8, about 0.2-about 1.5, about 0.3-about 1.5, about 0.4-about 1.3, or preferably about 0.5-about 1.0 μM of RA per day.

In one instance the culture is supplemented with about 5-about 15, preferably about 8-about 12 um DAPT per day.

In one instance the culture is supplemented with about 20 to about 40 ng/ml Triiodothyronine per day, about 8 to about 12 um DAPT per day and 0.5 to about 1.0 μM of RA per day.

In one aspect the invention provides a method of producing a synthetic retina, comprising differentiating a stem cell culture in a culture medium and supplementing the culture with retinoic acid and Triiodothyronine for a first time period from about day 50 to about day 150 of cell differentiation. The supplementation with retinoic acid and Triiodothyronine may be simultaneous, concomitant, sequential, or adjunctive.

In one instance said first time period is from about day 80 to about day 130, preferably from about day 90 to about day 120 of cell differentiation culture.

In one instance the method comprises additionally supplementing the differentiation culture with one or more of 9-cis-retinal, 11-cis retinal, Levodopa and DAPT.

In one instance said additionally supplementing comprises supplementing said differentiation culture with retinoic acid and Levodopa for a second time period. Said second time period may be from about day 50 to about day 150, from about day 80 to about day 130, or preferably from about day 90 to about day 120 of cell differentiation culture. The supplementation with retinoic acid and Levodopa may be simultaneous, concomitant, sequential, or adjunctive.

In one instance said additionally supplementing comprises supplementing the culture with retinoic acid and DAPT for a third time period. Said third time period may be from about day 10 to about day 150, from about day 20 to about day 130, or preferably from about day 30 to about day 120 of cell differentiation culture. The supplementation with retinoic acid and DAPT may be simultaneous, concomitant, sequential, or adjunctive.

In one instance said additionally supplementing comprises supplementing said culture with retinoic acid and Levodopa for a second time period and supplementing said culture with retinoic acid and DAPT for a third time period. Said second time period may be from about day 50 to about day 150 and said third time period may be from about day 10 to about day 150 of differentiation culture. Preferably, said second time period is from about day 90 to about day 120 of cell culture and said third time period is from about day 30 to about day 120 of cell differentiation culture.

In one instance said first time period is from about day 90 to about day 120 of cell differentiation culture, and/or said second time period is from about day 90 to about day 120 of cell culture and/or said third time period is from about day 30 to about day 120 of cell differentiation culture.

In one instance the end of culture is at about day 300, day 250, day 200, or day 150 of cell differentiation.

In a further aspect the invention provides a method of promoting rod formation and/or S cone formation in a stem cell differentiation culture comprising supplementing said culture with retinoic acid and Triiodothyronine for a period from about day 50 to about day 150 of cell differentiation culture. In one instance said period is from about day 80 to about day 130, preferably from about day 90 to about day 120 of cell culture. The supplementation with retinoic acid and Triiodothyronine may be simultaneous, concomitant, sequential, or adjunctive.

In one instance, the method further comprises supplementing said culture with retinoic acid and Levodopa. In one instance said period is from about day 50 to about day 150 of culture, from about day 80 to about day 130, preferably from about day 90 to about day 120 of cell culture.

In one instance, the method further comprise supplementing said culture with retinoic acid and DAPT for a period from about day 10 to about day 150 of culture, from about day 20 to about day 130, or preferably from about day 30 to about day 120 of cell culture. The supplementation with retinoic acid and DAPT may be simultaneous, concomitant, sequential, or adjunctive.

In one instance the end of culture is at about day 300, day 250, day 200, or day 150 of cell differentiation.

In a further aspect the invention provides a method of promoting S cone formation in a stem cell differentiation culture comprising supplementing said culture with retinoic acid and Levodopa for a period from about day 50 to about day 150 of culture differentiation culture. In one instance said period is from about day 80 to about day 130, preferably from about day 90 to about day 120 of cell culture. The supplementation with retinoic acid and Levedopa may be simultaneous, concomitant, sequential, or adjunctive.

In one instance, the method further comprise supplementing said culture with retinoic acid and DAPT for a period from about day 10 to about day 150 of culture, from about day 20 to about day 130, or preferably from about day 30 to about day 120 of cell culture.

In one instance the method further comprises supplementing said culture with retinoic acid and Triiodothyronine for a period from about day 50 to about day 150 of cell differentiation culture. In one instance said period is from about day 80 to about day 130, preferably from about day 90 to about day 120 of cell culture.

In one instance the end of culture is at about day 300, day 250, day 200, or day 150 of cell differentiation.

In a further aspect, the invention provides a method of promoting L cone and/or M cone formation in a stem cell differentiation culture comprising supplementing said culture with retinoic acid and DAPT for a period from about day 10 to about day 150 of culture. In one instance said period is from about day 20 to about day 130, or preferably from about day 30 to about day 120 of cell culture. The supplementation with retinoic acid and DAPT may be simultaneous, concomitant, sequential, or adjunctive.

In one instance the method further comprises supplementing said culture with retinoic acid and Triiodothyronine for a period from about day 50 to about day 150 of cell differentiation culture. In one instance said period is from about day 80 to about day 130, preferably from about day 90 to about day 120 of cell culture

In one instance, the method further comprises supplementing said culture with retinoic acid and Levodopa. In one instance said period is from about day 50 to about day 150 of culture, from about day 80 to about day 130, preferably from about day 90 to about day 120 of cell culture.

In one instance the end of culture is at about day 300, day 250, day 200, or day 150 of cell differentiation.

In a further aspect the invention provides a method of producing a synthetic retina, comprising differentiating a stem cell culture in a cell culture medium and supplementing the culture with:
(i) retinoic acid for a first time period from about day 10 of cell culture to about at least about 10 days prior to the end of differentiation, or until the end of differentiation;
(ii) Levodopa and/or Triiodothyronine for a second time period from about day 50 of cell culture to about at least about 10 days prior to the end of differentiation; and
(iii) DAPT for a third time period from about day 10 of cell culture to about at least about 10 days prior to the end of differentiation.

In so far as the invention relates to culture methods wherein for any aspect, said culture may be a three-dimensional stem cell culture, optionally wherein said three-dimensional stem cell culture is an embryoid body (EB) culture.

In all of the aforementioned aspects, the culture is supplemented with Retinoic Acid (RA) at a concentration of from about 0.1 to about 1 μM, preferably from about 0.3 to about 0.7 μM, more preferably at about 0.5 μM.

In all of the aforementioned aspects, the culture is supplemented with Levodopa at a concentration of from about 0.1 to about 1 μM, preferably from about 0.3 to about 0.7 μM, more preferably at about 0.5 μM.

In all of the aforementioned aspects, the culture is supplemented with DAPT at a concentration of from about 5 to about 15 μM, preferably from about 7 to about 12 μM, more preferably at about 10 μM.

In all of the aforementioned aspects, the culture is supplemented with Triiodothyronine (T3) at a concentration of from about 10 to about 60 ng/ml, preferably from about 20 to about 50 ng/ml, more preferably at about 40 ng/ml.

As used herein “three dimensional stem cell culture” refers to a cell culture having three-dimensional (3D) culture structure and is distinct from cultures grown on 2D substrates. Preferably, said three dimensional stem cell culture is embryoid body (EB) culture. The term “embryoid body” or “EB” refers to collections of cells formed from the aggregation or clustering of cultured embryonic stem cells in culture. EBs have a three-dimensional morphology, e. g., they can be a solid or a cystic embryoid body. Alternatively, the 3D stem cell culture may be grown within a bioreactor or spinner flask, as hanging drops from the lid of tissue culture vessels or within a scaffold or matrix, such as matrigel. Examples of such cultures are known in the art, such as Lancaster et al, Nature. 2013 Sep. 19; 501(7467):373-9. doi: 10.1038/nature12517. Epub 2013 Aug. 28.

In one embodiment the step of differentiating the stem cell culture is feeder free. Prior to differentiation, the three dimensional stem cell culture may be expanded either under feeder or feeder free conditions.

Alternative culture systems, compatible with the methods of the invention include three-dimensional culture for selective neural retinal differentiation by timed BMP4 treatment, followed by inhibiting GSK3 and FGFR as described in Kuwahara et al Nat Commun. 2015 Feb. 19; 6:6286. Similarly, three-dimensional optic vesicle structures may be produced using the NRL^{+/eGFP} hESC line and culture methods described in Philips et al Sci Rep. 2018 Feb. 5; 8(1):2370. Zhong et al Nat Commun. 2014 Jun. 10; 5:4047 and Gonzalez-Cordero et al Stem Cell Reports. 2017 Sep. 12; 9(3):820-837 also describe stem cell differentiation methods compatible with the present invention.

In one instance, the methods of the invention may involve a step of extracting of isolating said synthetic retina from said cell culture medium.

In one instance, the methods of the invention may involve a step of extracting at least one photoreceptor from said synthetic retina.

### Synthetic Retina

Emergence of the early synthetic retina is characterised by phase bright neuroepithelium (expressing Rax/Pax6) which evaginates from the edge of differentiating EB's, akin to the optic vesicles during normal development. This develops over time to form a bilayered optic cup. The inner wall of the cup contains retinal progenitors (expressing Pax6, Chx10) which subsequently differentiate into more mature retinal phenotypes and migrate to finally reside within distinct retinal neural layers, forming fully laminated retina (with different retinal phenotypes expressing Crx, Recoverin, Opsin, Calbindin 28, TUJ1, HuC/D and Islet1/2). The outer wall of the cup gives rise to retinal pigmented epithelium, characterised by its dark pigmentation and expression of RPE65 and ZO-1, which arises in parallel alongside neural retinal tissue.

In one instance, the differentiation is stopped when the synthetic retina comprising photoreceptors are formed. In one instance synthetic retina comprising photoreceptors are formed by about day 300, day 250, day 200, or day 150 of cell differentiation.

Methods of isolating photoreceptors from said synthetic retina are well known in the art, see for example Colin et al, Stem Cells 2019; 37:609-622.

### Medical Uses

The synthetic retinae and/or photoreceptors of the invention are of particular use in various therapeutic settings. In particular, the synthetic retinae are of particular use in transplantation and engraftment, for example to treat disease, injury or wounding.

The therapeutic benefice of transplanting photoreceptors, together with methods for transplanting photoreceptors, into subjects in need thereof, is well known in the art, see for example Colin et al, Stem Cells 2019; 37:609-622. Accordingly, the invention provides the use of the synthetic retinae and/or photoreceptors of the invention as a medicament.

The invention also provides a method of treating an ocular disease or injury comprising implanting a synthetic retina and/or photoreceptors of the invention into the eye of a mammalian subject in need thereof.

Generally, the ocular disease or injury is related to a damaged retina and/or retinal degenerative.

As used herein, the term “ocular injury” refers to conditions resulting in an insufficient stromal micro-environment to support stem cell function, for example aniridia, keratitis, neurotrophic keratopathy, Keratoconus, Meesman's dystrophy, Epithelial Basement Membrane Dystrophy and chronic limbitis; or conditions that destroy limbal stem cells such as Partial limbal stem cell deficiency, Total stem cell deficiency, Ocular herpes, chemical or thermal injuries, Stevens-Johnson syndrome, ocular cicatricial pemphigoid, contact lens wear, or microbial infection.

As used herein the term “retinal degenerative disease” refers to a disease or disorder selected from Retinitis Pigmentosa, age-related macular degeneration, Bardet-Biedel syndrome, Bassen-Kornzweig syndrome, Best disease, choroideremia, gyrate atrophy, Leber congenital amaurosis, Refsun syndrome, Stargardt disease or Usher syndrome.

Preferably, said synthetic retina and/or photoreceptors is derived from autologous cells, i.e. said cells are derived from the individual to be treated. Alternatively, the cells may be non-autologous.

Also provided is a method of retinal replacement, comprising implanting synthetic retina of the invention into the eye of a mammalian subject in need thereof. There is also provided synthetic retina of the invention for use in retinal replacement.

Also provided is a method of photoreceptor replacement, comprising implanting photoreceptors of the invention into the eye of a mammalian subject in need thereof.

There is also provided photoreceptors of the invention for use in photoreceptor replacement.

As used herein the terms “wound” and “wounding” relate to damaged tissues, preferably damaged retina, where the integrity of the retina or tissue is disrupted as a result of e.g. external force, bad health status, aging, exposure to sunlight, heat or chemical reaction or as a result from damage by internal physiological processes.

The synthetic retinae, photoreceptors and/or compositions of the invention may be placed into the interior of an eye using a syringe, a needle, a cannula, a catheter, a pressure applicator, and the like.

### Pharmaceutical Compositions

The invention also provides a pharmaceutical composition comprising a synthetic retina and/or photoreceptors in accordance with the invention together with a pharmaceutically acceptable excipient, dilutent or carrier.

In one embodiment the composition further comprises one or more of the following: growth factors, lipids, genes, etc., or compounds for altering the acidity/alkalinity (pH) of the wound, or compounds for altering the growth and performance of the transplanted cells and those at the margins of the wound/injury.

The term “pharmaceutically-acceptable carrier” as used herein means one or more compatible solid or liquid fillers, diluents or encapsulating substances that are suitable for administration into a human. When administered, the pharmaceutical compositions of the present invention are administered in pharmaceutically acceptable preparations. Such preparations may routinely contain pharmaceutically acceptable concentrations of salt, buffering agents, preservatives, compatible carriers, cytokines and optionally other therapeutic agents, preferably agents for use in wound healing such as growth factors, peptides, proteolytic inhibitors, extracellular matrix components, steroids and cytokines. The term “carrier” denotes an organic or inorganic ingredient, natural or synthetic, with which the active ingredient is combined to facilitate the application. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. As used herein, a pharmaceutically acceptable carrier includes any conventional carrier, such as those described in Remington's Pharmaceutical Sciences, by E. W. Martin, Mack Publishing Co, Easton, Pa., 15th Edition (1975).

In a further aspect there is provided a pharmaceutical composition in accordance with the invention for use as a medicament, for example, for use in treating ocular injury, retinal degeneration and/or injury.

The compositions or synthetic retinae of the invention are administered/for administration in effective amounts. An “effective amount” is the amount of a composition or synthetic retinae that alone, or together with further doses, produces the desired response. The compositions or synthetic retinae used in the foregoing methods preferably are sterile and contain an effective amount of the active ingredient for producing the desired response in a unit of weight or volume suitable for administration to a patient. The response can, for example, be measured by measuring the physiological effects of the composition or micro-organ cell composites upon the rate of or extent of disease treatment or retinal repair.

The invention provides a method to decrease the degeneration of a retina associated with a retinal degenerative disease or an ocular injury in a subject in need thereof comprising administration of a pharmaceutical composition of the invention to the subject in an amount effective to decrease further degeneration of the retina and/repair the retina.

### Models

The synthetic retinae and/or photoreceptors of the invention provide an in vitro model system. For example the synthetic retinae can be used as a model to provide insight into retinal and neurological development and also to provide insight into retinal and neurological disease development. The use of the synthetic retinae as a model for disease is useful for the diagnosis and treatment of retinal diseases and disorders. The aforementioned uses may involve retinal manipulations or disease modeling (for example induced by pharmacological treatment or genetic modifications) so as to develop drugs, or to diagnose, prevent, limit or cure retinal diseases and disorders.

The synthetic retina and/or photoreceptors of the invention also provide a screening method for identification of candidate molecules for the treatment of retinal diseases and disorders. This screening method is usable for example for preclinical testing of drugs to treat retinal diseases and disorders. Screening methods may be applied onto cell-based models of the present invention. Any suitable screening method known by the skilled person may be applied to the models of the invention.

The synthetic retinae and/or photoreceptors of the invention are useful in drug screening, for example, useful for identification of both direct and indirect pharmacologic agent effects. For example, certain candidate bioactive agents may be added to the model and the effect(s) monitored. For example, cell/cell interactions and cell/extracellular component interactions may be monitored as they may be important in understanding mechanisms of disease and drug function.

In one aspect the synthetic retinae and/or photoreceptors of the invention may be used in a method of screening a bioactive agent for the treatment of a retinal or ocular disease or disorder comprising: a) providing a synthetic retina in accordance with the invention; b) exposing said retina to an agent; and c) determining whether the agent has a therapeutic effect on the retina.

For example, the in vitro synthetic retinae and/or photoreceptors described herein may be used for identification and biological testing of bioactive agents and compounds, particularly neuroactive agents and compounds, or materials that may be suitable for treatment of neurological diseases or disorders in general, and in particular for treatment of retinal diseases and disorders. The described screening methods may be used to identify a bioactive agent that may be suitable for treating a subject who has a neurological, particularly, a retinal disease or disorder.

The screening methods may be used to determine if the bioactive agent alters (increases or decreases in a statistically significant manner) viability of a retinal cell. The screening method may also be used to determine whether the bioactive agent is capable of altering neurodegeneration of neuronal cells (impairing, inhibiting, preventing, abrogating, reducing, slowing the progression of, or accelerating in a statistically significant manner).

Preferably, said exposing comprises contacting (combining, mixing, or otherwise permitting interaction of) a candidate agent with the synthetic retinae under conditions and for a time sufficient to permit interaction between the candidate agent and the synthetic retinae, and then comparing the viability or degeneration of retinal cells in the presence of the candidate agent with the viability of retinal cells in the absence of the candidate agent. For example, retinal cells that are not exposed to a candidate agent may be prepared simultaneously, or alternatively, the effect of an agent may be quantified and compared to viability/degeneration of a standard retinal cell culture (i.e., a retinal cell culture system that provides repeatedly consistent, reliable, and precise determinations of retinal cell viability).

Through use of the synthetic retinae model described herein, agents may be selected and tested that are useful for treating diseases and disorders of the retina. More particularly, the presence of photoreceptors with an intact outer segment is relevant in such an assay to identify compounds useful for treating neurodegenerative eye diseases.

The synthetic retinae model of the invention may be used to screen candidate bioactive agents to determine whether the bioactive agent increases viability of retinal cells. A person skilled in the art would readily appreciate and understand that a retinal cell which exhibits increased viability means that the length of time that a retinal cell survives in the synthetic retinae system is increased (increased lifespan) and/or that the retinal cell maintains a biological or biochemical function (normal metabolism and organelle function; lack of apoptosis; etc.) compared with a retinal cell of a synthetic retinae of control cell system (e.g., the cell culture system described herein in the absence of the candidate agent). Increased viability of a retinal cell may be indicated by delayed cell death or a reduced number of dead or dying cells; maintenance of structure and/or morphology; lack of or delayed initiation of apoptosis; delay, inhibition, slowed progression, and/or abrogation of retinal neuronal cell neurodegeneration or delaying or abrogating or preventing the effects of neuronal cell injury. Methods and techniques for determining viability of a retinal cell and thus whether a retinal cell exhibits increased viability are described in greater detail herein and are known to persons skilled in the art.

For example a bioactive agent that inhibits may be screened by contacting (mixing, combining, the agent and the synthetic retina of the invention), for example, a candidate agent from a library of agents as described herein, with the model under conditions and for a time sufficient to permit interaction between a candidate agent and the synthetic retina as described herein.

### Experimental Methodology and Results

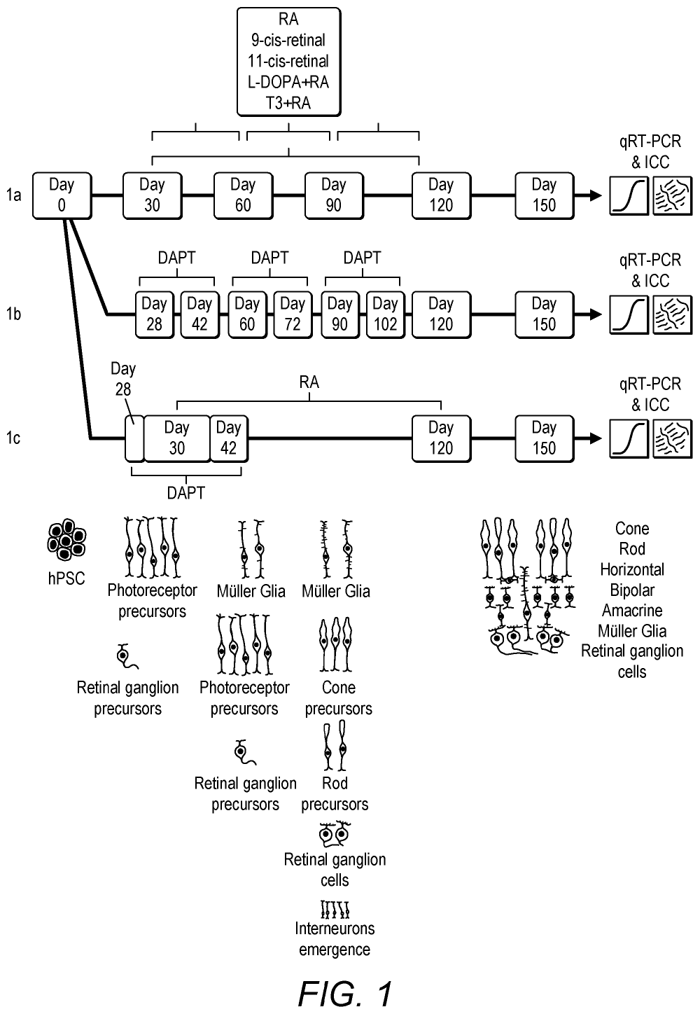

Human Pluripotent Stem Cells differentiation to retinal organoids Retinal organoids were differentiated from the CRX-GFP H9 human ESC cell line using a feeder-free system [24-25]. ESCs were expanded in mTeSR™1 (StemCell Technologies, 05850) at 37° C. and 5% CO2 on 6 well plates pre-coated with Low Growth Factor Matrigel (Corning, 354230). The retinal organoids were generated following a protocol described in Collin et al., 2019 [25] with addition of various supplements, which are shown in FIG. 1. The composition of the basal media for each stage of differentiation is shown in the Table 1.

The tested supplements include Retinoic Acid (RA) (0.5 μM or 1 μM Sigma-Aldrich UK), 9-cis-retinal (0.5 μM, Sigma-Aldrich UK), 11-cis-retinal (0.5 μM, BOC Sciences), Levodopa (0.5 μM, Sigma-Aldrich UK), Triiodothyronine (T3) (20 ng/ml or 40 ng/ml, Sigma-Aldrich) and DAPT (10 μM Sigma-Aldrich UK) singly or in combination with each other from day 18 of differentiation for specific durations (day 18 onwards, day 30-60, day 60-90, day 90-120 and day 30-120), except for DAPT (FIG. 1). DAPT was only added from day 30-42 (for day 30-60 combined addition with RA from day 30-60), day 60-72 (for the combined addition with RA from day 60-90), day 90-102 (for the combined addition with RA from day 90-120) and day 30-42 (for the combined addition with RA from day 30-120).

The inventors surprisingly identified that culture could supplemented to preferentially promote rod formation by the addition of T3 (20 ng/ml or 40 ng/ml) from day 18 of differentiation onwards together with the addition of RA (0.5 μM or 1 μM) from day 90-120 of differentiation.

The inventors surprisingly identified that culture could supplemented to preferentially promote cone formation (S and M/L cones) by the addition of T3 (20 ng/ml or 40 ng/ml) from day 18 of differentiation onwards, the addition of RA (0.5 μM or 1 μM) from day 30-120 of differentiation and the addition of DAPT from day 28-42 of differentiation.

### Immunohistochemistry

Retinal organoids were collected on day 150 and fixed in 2% PFA for 30 minutes, followed by three washes in Phosphate-buffered saline (PBS), incubated overnight in 30% sucrose in PBS, embedded in Optical Cutting medium (OCT) (Cellpath, UK) and frozen at −20° C. Ten-micrometre cryostat sections were collected using a Leica Cm1860 cryostat (Leica, Germany) onto Superfrost Plus slides and stored at −20° C. in slide boxes prior to immunostaining. Cryosections were air-dried, washed several times in PBS and incubated in blocking solution (10% normal goat serum, 0.3% Triton-X-100 in PBS) for one hour at room temperature. Slides were incubated with the appropriate primary antibody overnight at 4° C. (Table 2). After rinsing with PBS, sections were incubated with the secondary antibody for 2 hours at room temperature. Alexa Fluor 647 and 546 secondary antibodies (InvitrogenMolecular Probes) were used at a 1:1000 dilution. Negative controls were carried out by omitting the primary antibody. Afterwards, sections were washed three times in PBS and mounted with Vectashield (Vector Laboratories, Burlingame, Calif.) containing Hoechst (LifeTechnologies, UK).

### Image Acquisition and Analysis

Images were captured using an Axio Imager upright microscope with Apotome (Zeiss, Germany) structured illumination fluorescence using 20× objective and 63× oil objective operated with AxioVision software. Final images are presented as a maximum projection and adjusted for brightness and contrast in Adobe Photoshop (Adobe Systems).

### Image Quantification

Cell image quantitation was performed using the MATLAB software (Mathworks, MA) following the protocol described in Dorgau et al., 2019 [26]. A minimum of eight individual retinal organoids were analysed for each condition. All results were further analysed using Microsoft Excel and Prism (GraphPad, USA).

qRT-PCR

Retinal organoids were collected on day 150 and assessed by quantitative RT-PCR; 15-20 retinal organoids for each condition were homogenised using a Dounce Tissue Grinder (Sigma-Aldrich, UK) to extract the RNA using the Promega tissue extraction kit (Promega, USA) as per the manufactures instructions. 1 μg of RNA was reverse transcribed using random primers (Promega, USA). qRT-PCR was performed using a Quant Studio 7 Flex system (Applied Biosystems, USA) with SYBR Green reaction mixture (Promega, USA). Each primer (Table 3) was used at a concentration of 0.5 μM. The reaction parameters were as follows: 95° C. for 15 minutes to denature the cDNA and primers, 40 cycles of 94° C. for 15 seconds followed by primer specific annealing temperature for 30 seconds (60° C.), succeeded by a melt curve. A comparative cycle threshold (Ct) method was used to calculate the levels of relative expression, whereby the Ct was normalised to the endogenous control (GAPDH). This calculation gives the Δ^{Ct} value, which was then normalised to a reference sample (i.e. control group), giving the ΔΔ^{Ct}. The fold change was calculated using the following formula: 2-ΔΔ^{Ct}.

### Statistical Analyses

All statistical tests were performed using Prism (GraphPad, USA). The standard errors of all means (SEM) were calculated. Statistical significance was tested using either one-way ANOVA or two-way ANOVA (Dunnett statistical hypothesis for multiple test correction). Asterisk=p-value <0.05, two asterisks=p-value <0.01, three asterisks=p-value <0.001, four asterisks=p-value <0.0001.

### Results and Discussion

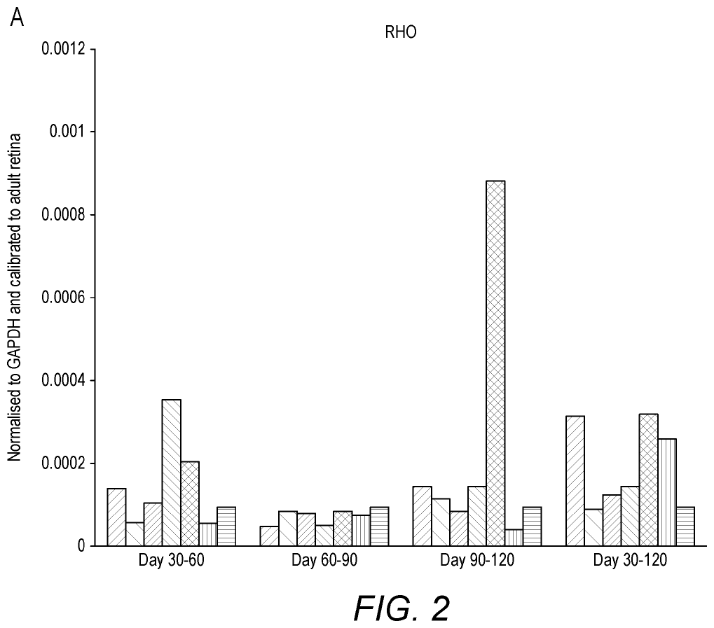

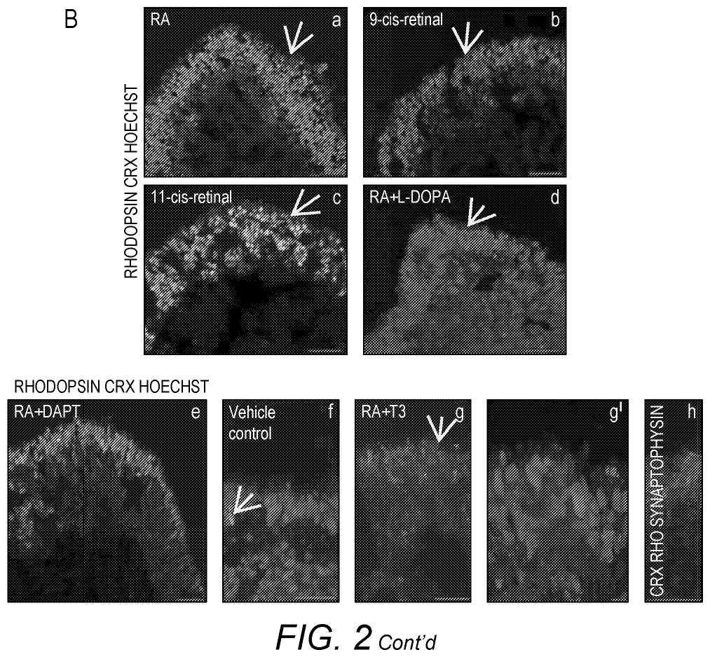

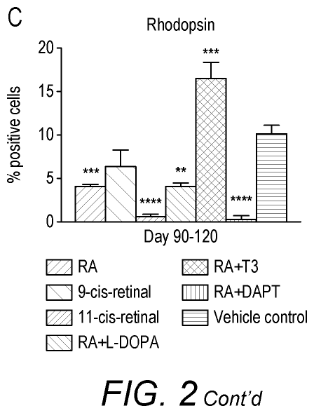

The CRX-GFP (H9) hESC line was expanded and differentiated to retinal organoids, which were collected at day 150 and processed for quantitative RT-PCR (qRT-PCR) and immunohistochemistry (IHC). The following reagents: Retinoic acid (RA), 9-cis-retinal, 11-cis-retinal, Levodopa (L-DOPA), Triiodothyronine (T3) and DAPT were added at specific time intervals during differentiation as shown in FIG. 1. All these supplements have been shown to promote cone or rod photoreceptor formation in other species. For example, it was shown that RA enhances rod differentiation in vivo and in vitro [10,11], expression of rod photoreceptor transcription factor NRL [12] and red cone opsin development in vivo [13]. T3 is also required for rod photoreceptor development in vivo [14], for regulating the ratio and patterning of cone opsin expression [15], specifying cone subtype generation [16] as well as suppressing cone viability [17]. In accordance with these published data the combined addition of RA and T3 during day 90-120 of differentiation or the addition of T3 from day 18 onwards of differentiation in combination with the addition of RA from day 90-120 of differentiation resulted in the highest gene expression of Rhodopsin (rod marker) (FIG. 2A). These results were further corroborated by the immunohistochemical analysis, which revealed a significantly higher number of rods (identified by the marker protein Rhodopsin), compared to the control group (vehicle alone) and the groups supplemented with 9- or 11-cis retinal, RA, RA+L-DOPA or RA+DAPT (FIG. 2B a-f,2C and FIG. 5). 9-cis-retinal has been shown to encourage rod differentiation in developing mammalian retina [18,19]. 11-cis-retinal, the light-sensitive component of rod and cone photoreceptors, has been shown to activate Rhodopsin expression in vitro [20]. Despite these findings, the present data show that the number of Rhodopsin positive cells were lower in RA alone, 9- and 11-cis retinal (FIG. 2B a-f and 2C) when compared to the control group. In all conditions, rods were located in the apical layer of the retinal organoids, forming a putative outer nuclear layer (ONL): furthermore, higher magnification observations indicated the typical morphology of mature rods (FIG. 2B g-g′). Synaptic connections between mature rod photoreceptors and second order neurons (Horizontal and Bipolar cells) within the outer plexiform layer (OPL) were observed by double staining with Rhodopsin and Synaptophysin, a marker for synapses, showing Synaptophysin expression underneath the ONL of retinal organoids (FIG. 2B h and FIG. 5).

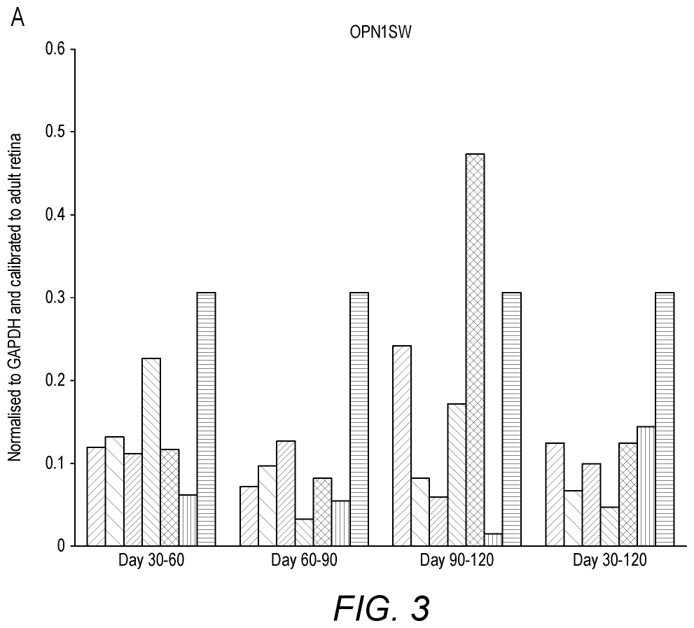

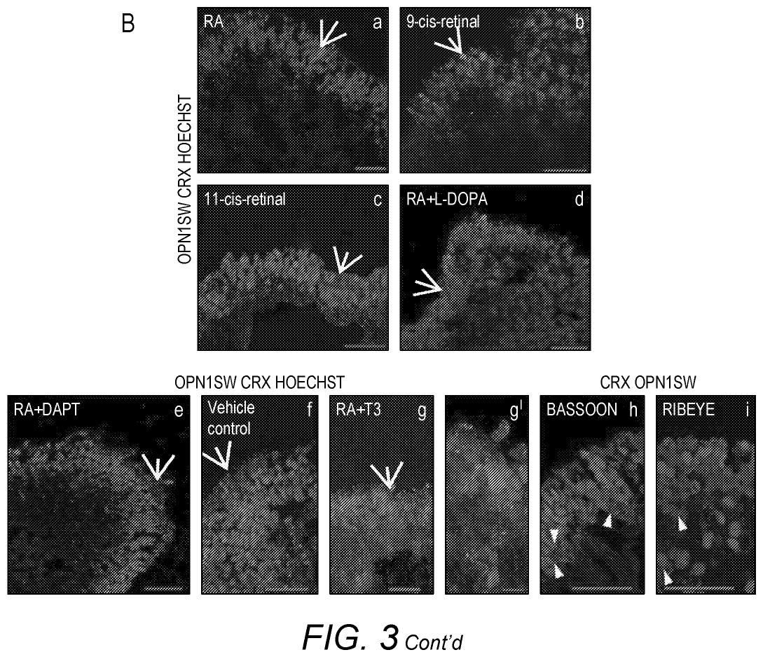

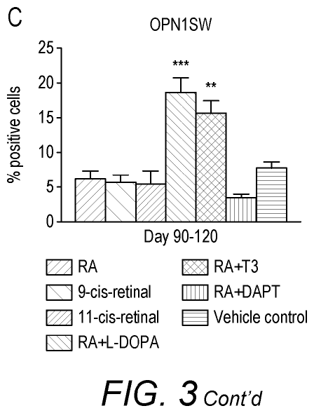

Addition of RA and T3 also resulted in the highest expression of S cone photoreceptor marker (OPN1SW) (FIG. 3A). Immunohistochemical analysis confirmed these findings, but also highlighted another group (RA+L-DOPA) to be equally good for the generation of S cones (FIG. 3B-C, FIGS. 6 and 7). L-DOPA is produced by the retinal pigment epithelium: its absence results in reduced rod numbers with no effect on the cone population in mouse ESC (mESC) [21]. In contrast, the inventors results in the human system revealed that the addition of RA+L-DOPA during day 90-120 of differentiation led to the enhancement of S-cone formation at the expense of rods (FIGS. 3C and 7). In this group, possible synapse formation between OPN1SW+ cells and second order neurons within the putative developing OPL was confirmed by the expression of Bassoon, an essential component of the ribbon synapses (FIG. 3B h arrowheads) and Ribeye, the main protein component of synaptic ribbons (FIG. 3B i arrowheads).

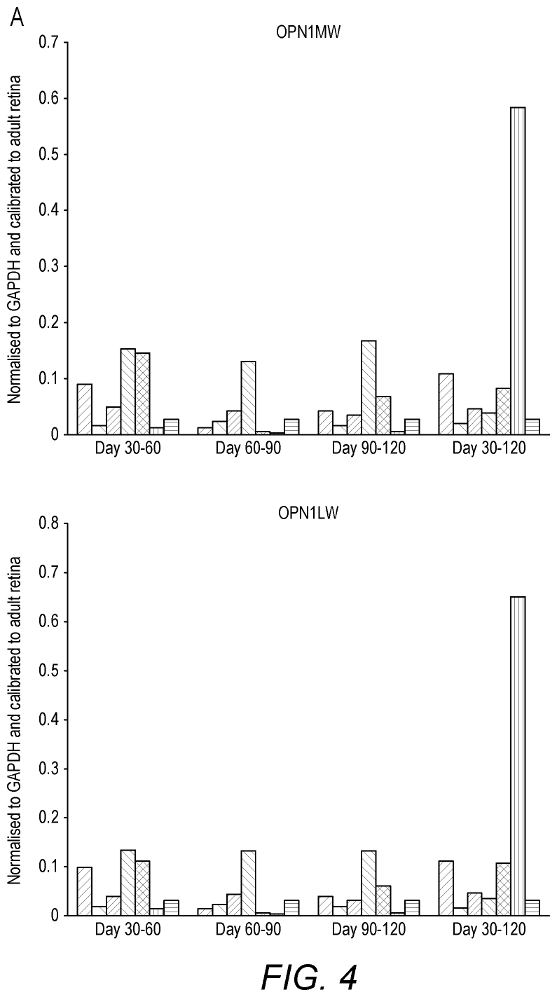

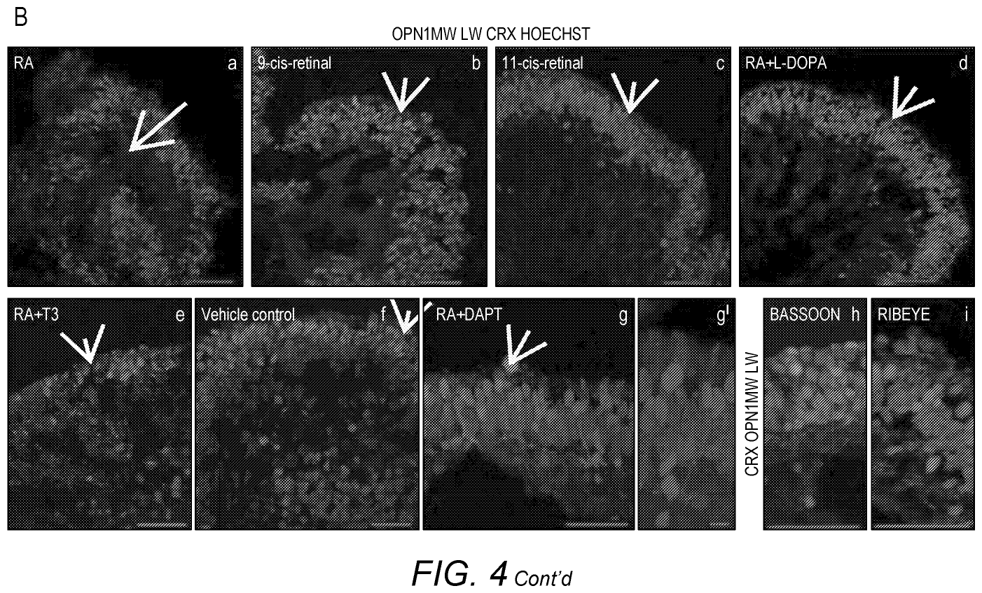

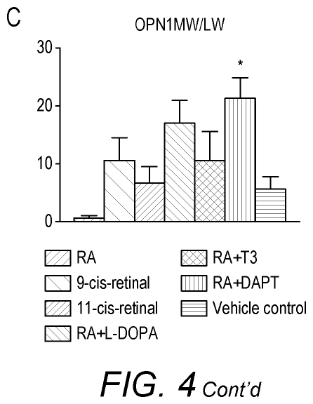

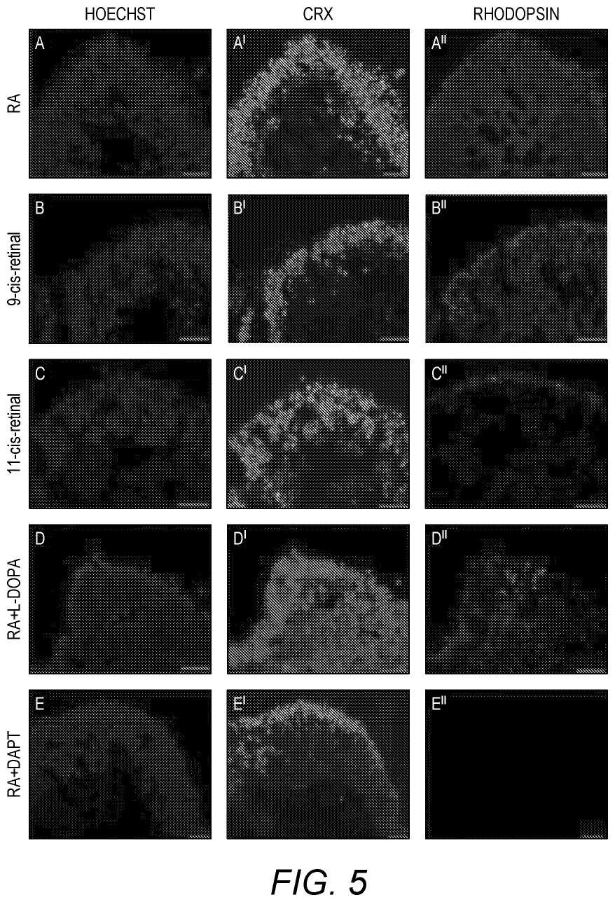

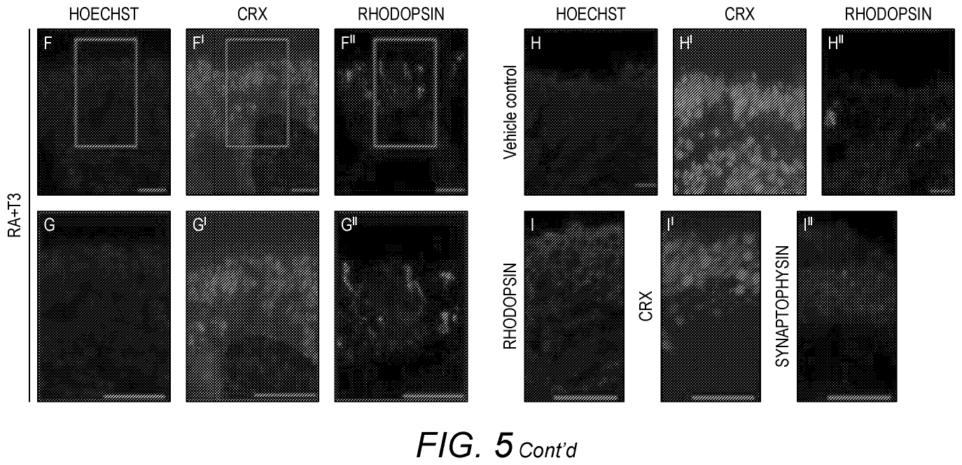

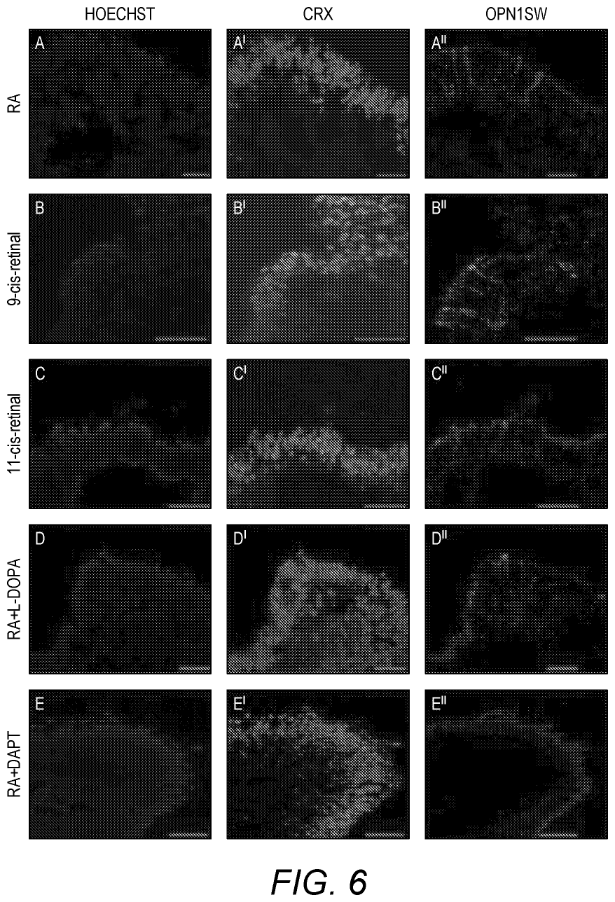

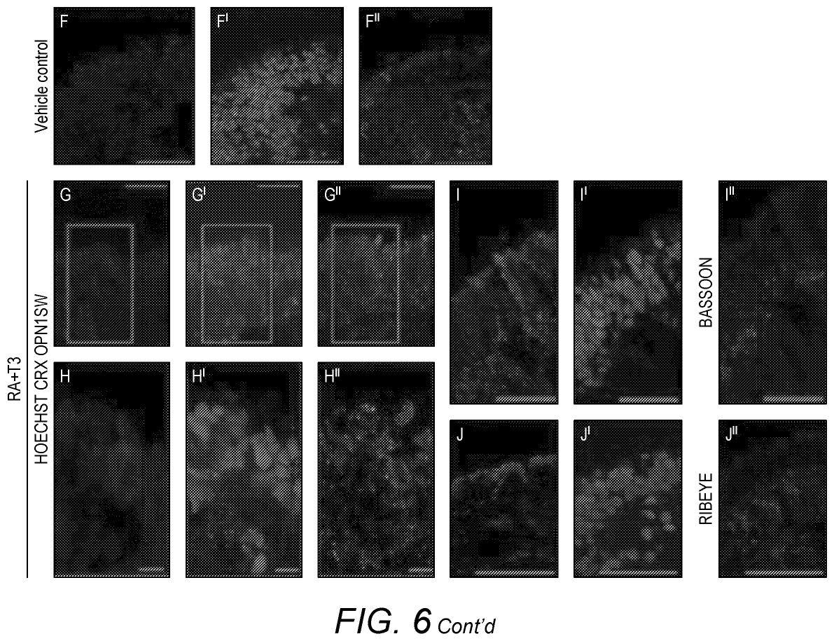

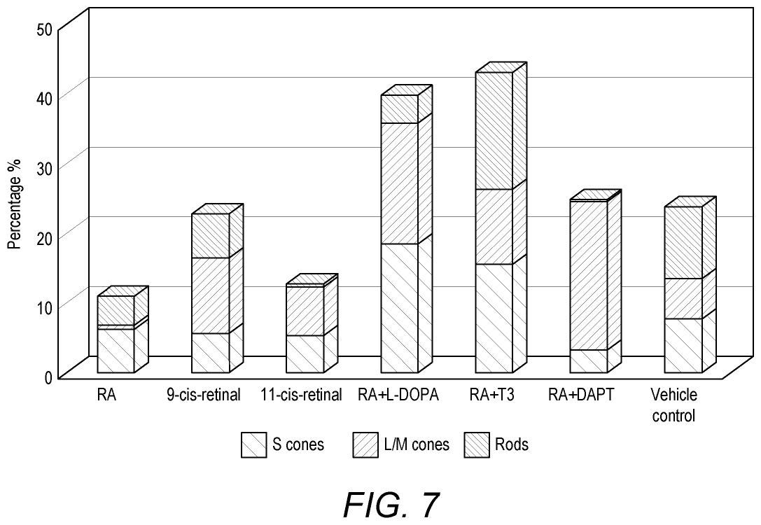

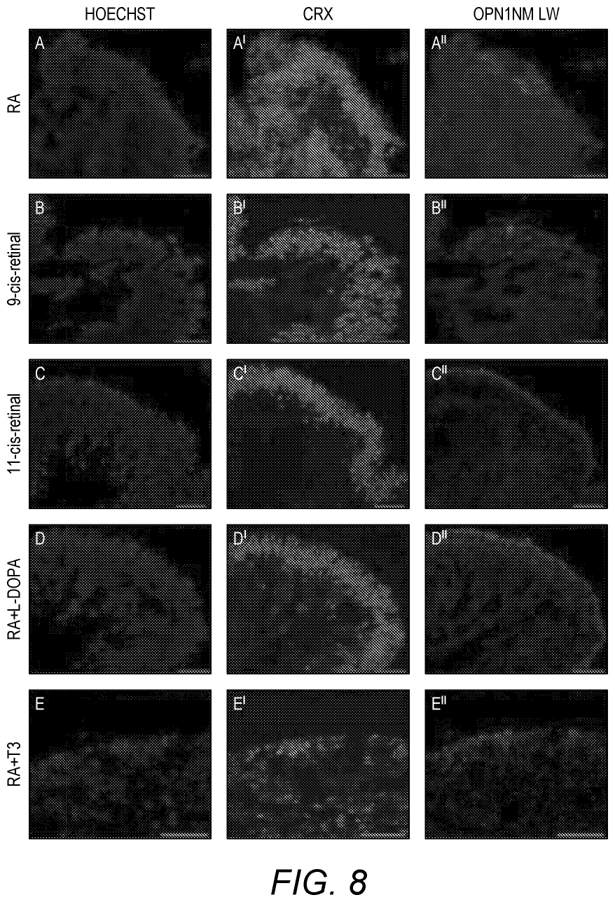

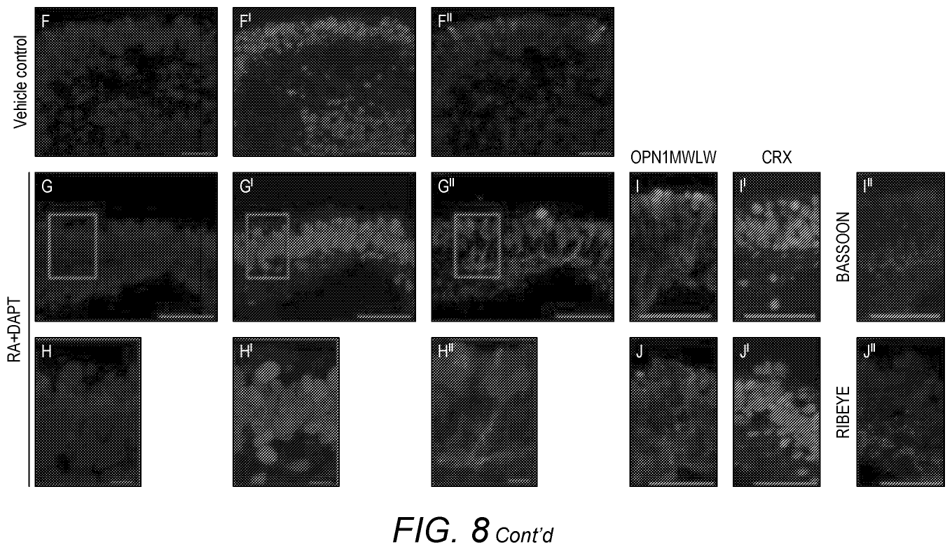

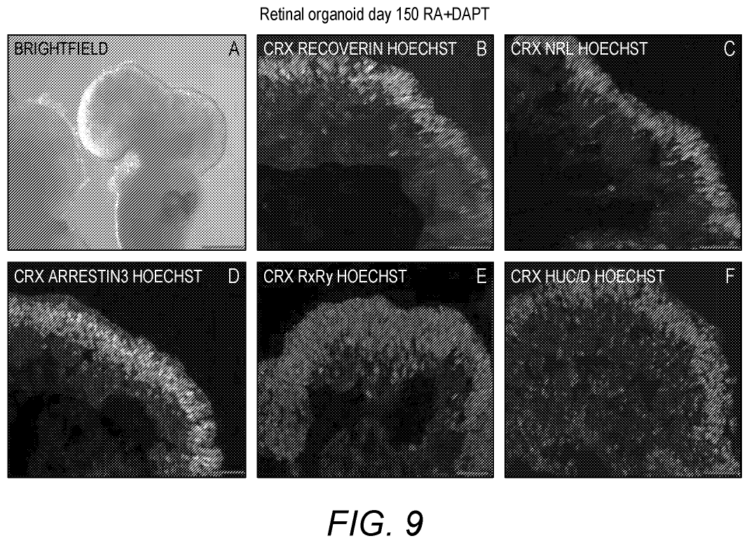

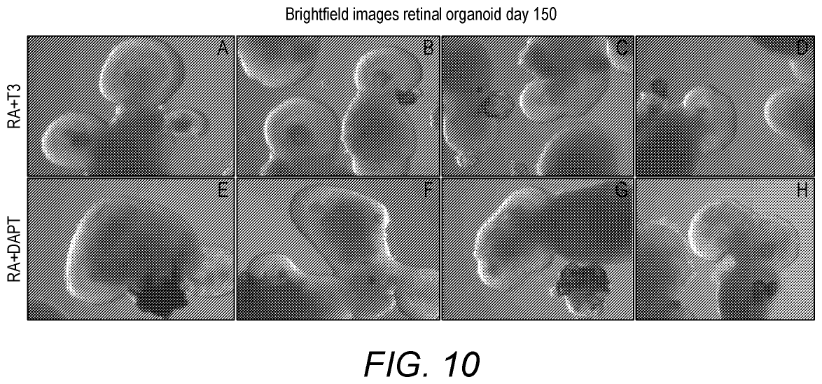

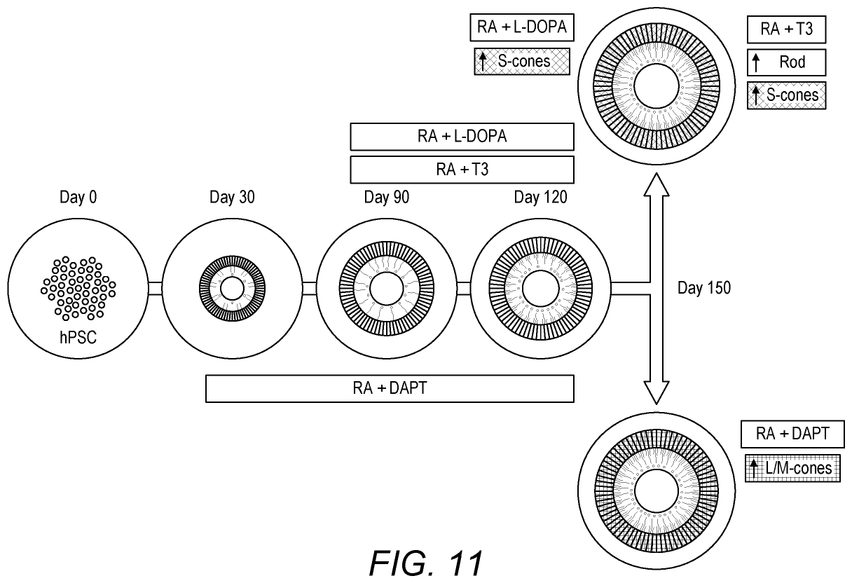

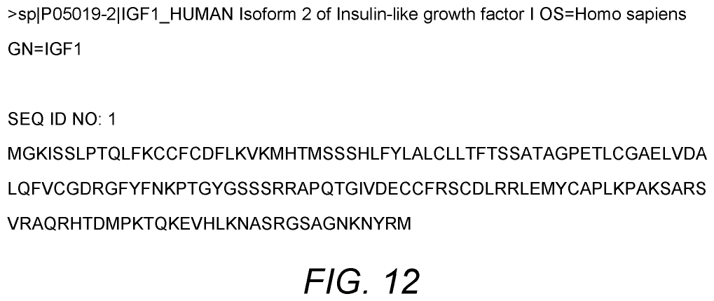

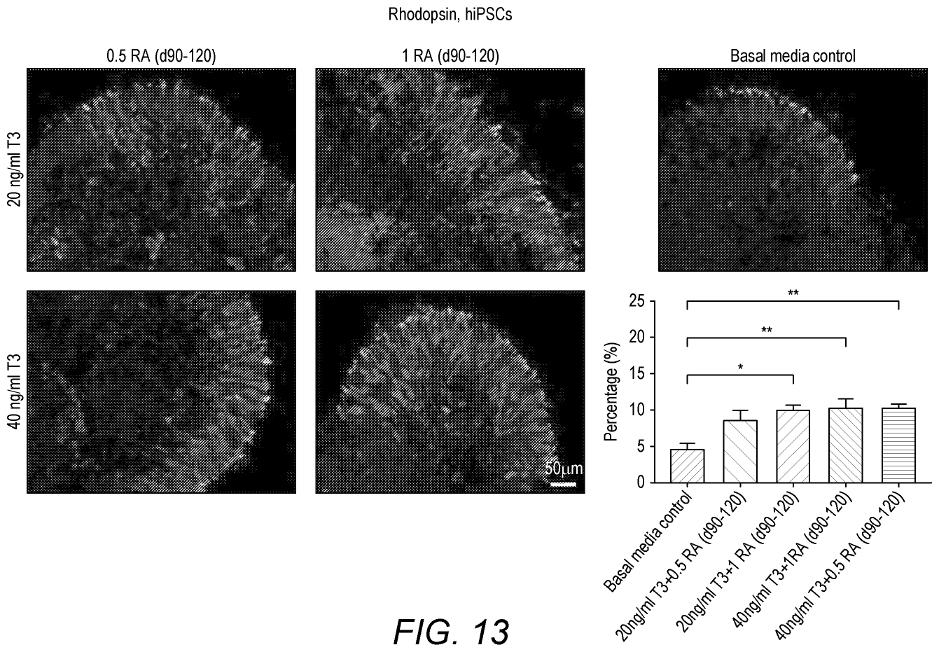

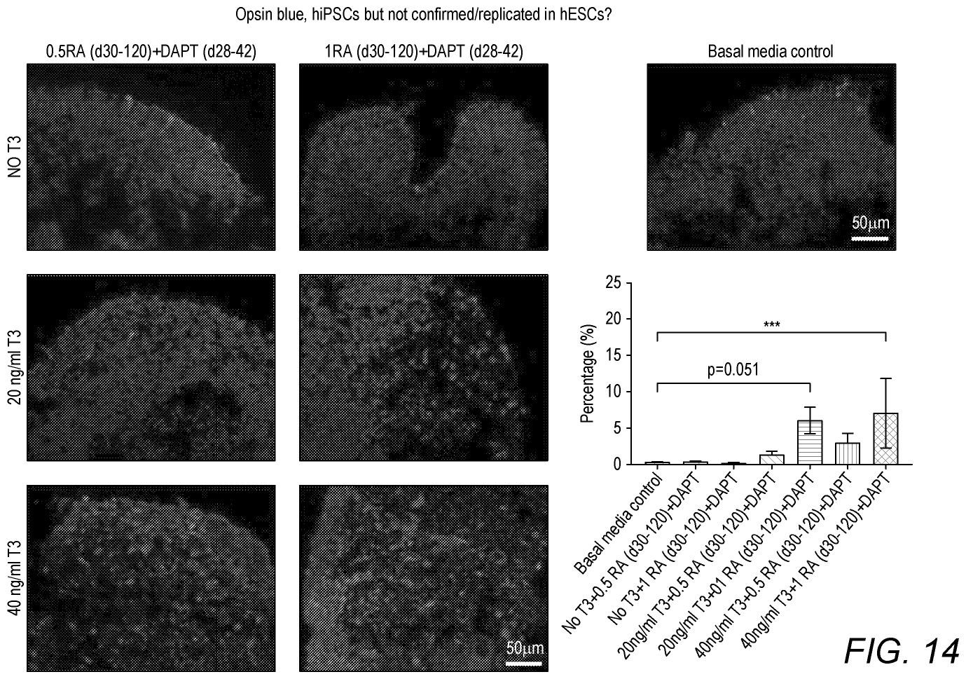

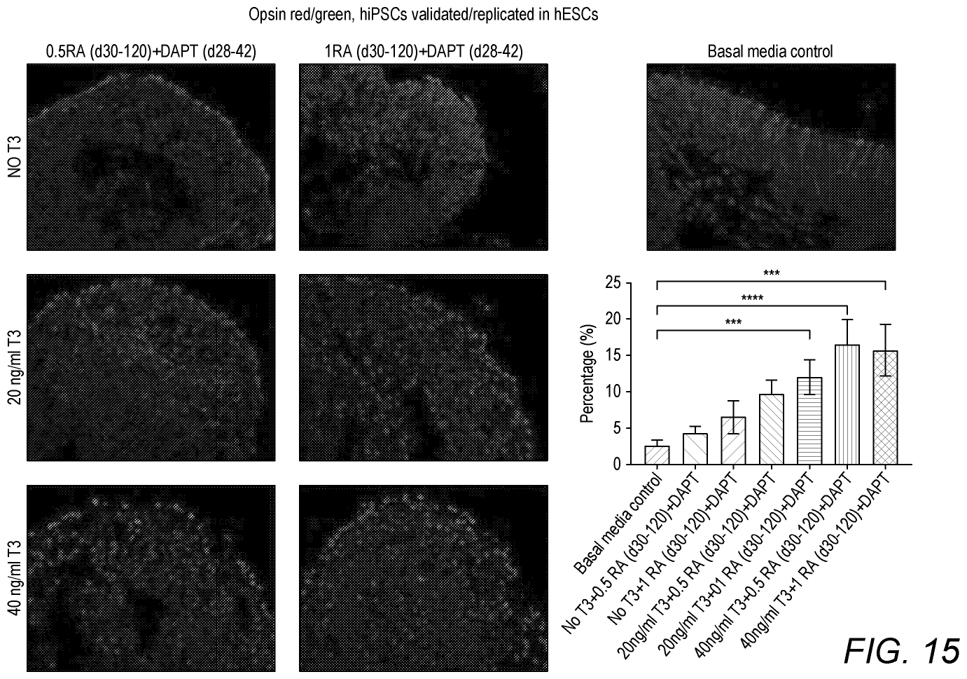

The gamma-secretase inhibitor DAPT, a known Notch signalling inhibitor, has been reported to increase cone or rod cell differentiation when added at specific stages of mESC differentiation [22,23]. Our qRT-PCR analysis showed the highest expression of OPN1MW (M cone photoreceptor marker) and OPN1LW (L cone photoreceptor marker) in the retinal organoids that had been exposed to the combined addition of RA+DAPT from day 30 to 120 of differentiation (FIG. 4A) and or the addition of T3 from day 18 onwards of differentiation in combination with the addition of DAPT from day 28 to 42 of differentiation and RA from day 90-120 of differentiation. Additionally, immunohistochemical analysis and its quantification indicated a significant increase in the percentage of mature L/M cones in this group at the expense of rods compared to the vehicle control (FIG. 4B a-g′, 4C, FIG. 7 and FIG. 8). In comparison to the vehicle control alone, all culture conditions revealed a slightly higher expression of OPN1MW/LW+ cells, except for the RA condition (FIG. 4C). As shown in Figure, OPN1MWLW expression was significantly enhanced upon addition of RA from day 30-120 of differentiation. Furthermore, the formation of cone pedicles in the putative OPL, where they form ribbon synapses with Horizontal/Bipolar cells, was demonstrated by the immunoreactivity of Bassoon and Ribeye respectively (FIG. 4B h-i).

These results were further corroborated by the immunohistochemical analysis in both hESCs and hiPSCs, which revealed a significantly higher number of cone photoreceptors (identified by the marker protein opsin blue), compared to the control group (vehicle alone) and the groups supplemented with 0.5 RA (days 30-120)+DAPT (days 28-42) and no T3; 0.5 RA (days 30-120)+DAPT (days 28-42) and 20 ng/mg T3 (day 18 onwards); 0.5 RA (days 30-120)+DAPT (days 28-42) and 40 ng/mg T3 (day 18 onwards); 1 RA (days 30-120)+DAPT (days 28-42) and no T3; 1 RA (days 30-120)+DAPT (days 28-42) and 20 ng/mg T3 (day 18 onwards); 1 RA (days 30-120)+DAPT (days 28-42) and 40 ng/mg T3 (day 18 onwards) (FIGS. 14 and 15).

Interestingly, in contrast to Nakano et al 2012 [6], the DAPT treatment did not interfere with the tissue architecture and the lamination of the retinal organoids (FIGS. 9 and 10). More extensive comparisons with work performed by other groups on the impacts of DAPT on retinal cell formation and maturation are not possible, as different groups use different media compositions for the culture of retinal organoids. The data demonstrate that addition of RA and T3 results in generation of all cone subtypes as well as rods at day 150 of differentiation, unlike the Eldred et al. 2018 study where by day 150 of differentiation only S cones were found within the retinal organoids. [16].

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

TABLE 1

| | Initial | | Final | | Day | Day | day | day |
|---|---|---|---|---|---|---|---|---|
| Product | Concentration | | Concentration | | 0-1 | 2-17 | 18-25 | 30-150 |
| mTeSR1 | | | | | ✓ | | | |
| Rock Inhibitor | 10 | mM | 10 | μM | ✓ | | | |
| DMEM/F12-Glutamax | | | | | | ✓ | ✓ | ✓ |
| KnockOut ™ Serum | | | 10% | | | ✓ | | |
| Replacement | | | | | | | | |
| Penicillin/ | 100x | | 1X | | | ✓ | ✓ | ✓ |
| Streptomycin | | | | | | | | |
| Fungizone | 250 | μg/ml | 25 | μg/ml | | | ✓ | ✓ |
| B27 | 50x | | 1X | | | ✓ | ✓ | ✓ |
| IGF-1 | 100 | μg/ml | 5 | ng/ml | | ✓ | ✓ | ✓ |

| NEEA | 100x | 1X | | ✓ | ✓ | ✓ |
|---|---|---|---|---|---|---|
| N2 | 100x | 1X | | | | ✓ |
| Taurine | 0.1M | 0.1 | mM | | ✓ | ✓ |

| | | | |
|---|---|---|---|
| Lipids | 100x | 1X | ✓ |

TABLE 2

| Antibody | Host | Dilution | Supplier, Cat. No |
|---|---|---|---|
| Anti-opsin Blue | Rabbit | 1:200 | Millipore, AB5407 |
| (OPN1SW) | | | |
| Anti-opsin Red/Green | Rabbit | 1:200 | Millipore, AB5405 |
| (OPN1LW/MW) | | | |
| Anti-RetP1(Rhodopsin) | Mouse | 1:200 | Sigma, O4886 |
| Anti-Bassoon | Mouse | 1:100 | StressGen, VAM-PS003 |
| Anti-Ribeye | Mouse | 1:100 | BD Bioscience, 612044 |
| Anti-Synaptophysin | Rabbit | 1:200 | abcam, ab32127 |

TABLE 3

| | Forward Primer | Reverse Primer | NCBI Referene |
|---|---|---|---|
| Gene | (Sequence (5′→3′) | (Sequence (5′→3′) | Sequence |
| GAPDH | TGCACCACCAACTGCTTAGC | GGCATGGACTGTGGTCATGAG | NM_001256799.2 |
| OPSINLW | GCCTACTTTGCCAAAAGTGC | GATGAGACCTCCGTTTTGGA | NM_020061 |
| OPSINMW | CATCTTTGGTTGGAGCAGGTACT | TCTCTGCCTTCTGGGTGGAT | NM_000513.2 |
| OPSINSW | ATACCGCAGCGAGTCCTATAC | GATCCTACCATCACAACCAC | NM_001708.2 |
| RHO | TTTGGAGGGCTTCTTTGCCA | CCTCGGGGATGTACCTGGAC | NM_000539.3 |

### REFERENCES

1 Chichagova V, Hallam D, Collin J, et al. Cellular regeneration strategies for macular degeneration: past, present and future. Eye 2018; 32:946-971.
2 Santos-Ferreira T F, Borsch O, Ader M. Rebuilding the Missing Part—A Review on Photoreceptor Transplantation. Front Syst Neurosci 2017; 10:105.
3 Thomson J A, Itskovitz-Eldor J, Shapiro S S, et al. Embryonic stem cell lines derived from human blastocysts. Science 1998; 282:1145-1147.
4 Takahashi K, Tanabe K, Ohnuki M, et al. Induction of Pluripotent Stem Cells from Adult Human Fibroblasts by Defined Factors. Cell 2007; 131:861-872.
5 Mellough C B, Collin J, Sernagor E, et al. Lab generated retina: realizing the dream. Vis Neurosci 2014; 31:317-332.
6 Nakano T, Ando S, Takata N, et al. Self-formation of optic cups and storable stratified neural retina from human ESCs. Cell Stem Cell 2012; 10:771-785.
7 Eiraku M, Sasai Y. Mouse embryonic stem cell culture for generation of three-dimensional retinal and cortical tissues. Nat Protoc 2012; 7:69-79.
8 Llonch S, Carido M, Ader M. Organoid technology for retinal repair. Dev Biol 2018; 433:132-143.
9 Jayakody S A, Gonzalez-Cordero A, Ali R R, et al. Cellular strategies for retinal repair by photoreceptor replacement. Prog Retin Eye Res 2015; 46:31-66.
10 Kelley M W, Turner J K, Reh T A. Retinoic acid promotes differentiation of photoreceptors in vitro. Development 1994; 120:2091-2102.
11 Kelley M W, Williams R C, Turner J K, et al. Retinoic acid promotes rod photoreceptor differentiation in rat retina in vivo. Neuroreport 1999; 10:2389-2394.
12 Khanna H, Akimoto M, Siffroi-Fernandez S, et al. Retinoic acid regulates the expression of photoreceptor transcription factor NRL. J Biol Chem 2006; 281:27327-27334.
13 Prabhudesai S N, Cameron D A, Stenkamp D L. Targeted effects of retinoic acid signaling upon photoreceptor development in zebrafish. Dev Biol 2005; 287:157-167.
14 Sawant O, Horton A M, Shukla M, et al. Light-Regulated Thyroid Hormone Signaling Is Required for Rod Photoreceptor Development in the Mouse Retina. Invest Ophthalmol Vis Sci 2015; 56:8248-8257.
15 Roberts M R, Srinivas M, Forrest D, et al. Making the gradient: thyroid hormone regulates cone opsin expression in the developing mouse retina. Proc Natl Acad Sci USA 2006; 103:6218-6223.
16 Eldred K C, Hadyniak S E, Hussey K A, et al. Thyroid hormone signaling specifies cone subtypes in human retinal organoids. Science (80-) 2018; 362:eaau6348.
17 Ma H, Ding X-Q. Thyroid Hormone Signaling and Cone Photoreceptor Viability 2016:613-618.
18 Söderpalm A K, Fox D A, Karlsson J O, et al. Retinoic acid produces rod photoreceptor selective apoptosis in developing mammalian retina. Invest Ophthalmol Vis Sci 2000; 41:937-947.
19 Wallace V A, Jensen A M. IBMX, taurine and 9-cis retinoic acid all act to accelerate rhodopsin expression in postmitotic cells. Exp Eye Res 1999; 69:617-627.
20 Kono M, Goletz P W, Crouch R K. 11-cis- and all-trans-retinols can activate rod opsin: rational design of the visual cycle. Biochemistry 2008; 47:7567-7571.
21 Jeffery G. The retinal pigment epithelium as a developmental regulator of the neural retina. Eye (Lond) 1998; 12 (Pt 3b):499-503.
22 Kruczek K, Gonzalez-Cordero A, Goh D, et al. Differentiation and Transplantation of Embryonic Stem Cell-Derived Cone Photoreceptors into a Mouse Model of End-Stage Retinal Degeneration. Stem Cell Reports 2017; 8:1659-1674.
23 Völkner M, Zschätzsch M, Rostovskaya M, et al. Retinal Organoids from Pluripotent Stem Cells Efficiently Recapitulate Retinogenesis. Stem Cell Reports 2016; 6:525-538.
24 Collin J, Mellough C B, Dorgau B, et al. Using Zinc Finger Nuclease Technology to Generate CRX-Reporter Human Embryonic Stem Cells as a Tool to Identify and Study the Emergence of Photoreceptors Precursors During Pluripotent Stem Cell Differentiation. Stem Cells 2016; 34:311-321.
24 Collin J, Queen R, Zerti D, et al. Deconstructing Retinal Organoids: Single Cell RNA-SeqReveals the Cellular Components of Human Pluripotent Stem Cell-Derived Retina. Stem Cells 2019.
25 Dorgau B, Felemban M, Hilgen G, et al. Decellularised extracellular matrix-derived peptides from neural retina and retinal pigment epithelium enhance the expression of synaptic markers and light responsiveness of human pluripotent stem cell derived retinal organoids. Biomaterials 2019; 199:63-75.

1. A method of producing a synthetic retina, comprising differentiating a stem cell culture in a culture medium and supplementing the culture with:
(i) Triiodothyronine from about day 18 of cell differentiation;
(ii) DAPT from about day 28 to about day 42 of cell differentiation; and
(iii) retinoic acid for a first time period from about day 30 to about day 120 of cell differentiation.

2. (canceled)

3. The method of claim 1, wherein said culture medium is a three-dimensional stem cell culture or a neural cell culture medium.

4. The method of claim 1, wherein said culture media is supplemented with an IGF-1 receptor agonist selected from the group consisting of:
i) a human IGF-1;
ii) a homologue of human IGF-1; and
iii) an analogue of human IGF-1.

5. The method of claim 1, further comprising supplementing the differentiation culture with one or more selected from the group consisting of 9-cis-retinal, 11-cis retinal, and Levodopa.

6. The method of claim 5, wherein said supplementing comprises supplementing said differentiation culture with retinoic acid and Levodopa for a second time period, and wherein said second time period is from about day 50 to about day 150.

7. (canceled)

8. A method of producing a synthetic retina, comprising differentiating a stem cell culture in a culture medium and supplementing the culture with:
(i) Triiodothyronine from about day 18 of cell differentiation; and
(ii) retinoic acid for a first time period from about day 90 to about day 120 of cell differentiation.

9. (canceled)

10. The method of claim 8, wherein said culture medium is a neural cell culture medium or a three-dimensional stem cell culture.

11. The method of claim 8, wherein said culture media is supplemented with an IGF-1 receptor agonist selected from the group consisting of:
i) a human IGF-1;
ii) a homologue of human IGF-1; and
iii) an analogue of human IGF-1.

12. The method of claim 8, further comprising supplementing the differentiation culture with one or more selected from the group consisting of 9-cis-retinal, 11-cis retinal, Levodopa and DAPT.

13. The method of claim 12, wherein said supplementing comprises supplementing said differentiation culture with retinoic acid and Levodopa for a second time period, and wherein said second time period is from about day 50 to about day 150.

14. (canceled)

15. The method of claim 12, wherein said additionally supplementing comprises supplementing the culture with retinoic acid and DAPT for a third time period, and wherein said third time period is from about day 10 to about day 150.

16. (canceled)

17. The method of claim 8, further comprising supplementing said culture with retinoic acid and Levodopa for a second time period and supplementing said culture with retinoic acid and DAPT for a third time period, and wherein said second time period is from about day 90 to about day 120 of cell culture and said third time period is from about day 30 to about day 120 of cell differentiation culture.

18. (canceled)

19. A method of promoting rod formation in a stem cell differentiation culture comprising supplementing the culture with:
(i) Triiodothyronine from about day 18 of cell differentiation; and
(ii) retinoic acid for a first time period from about day 90 to about day 120 of cell differentiation.

20. A method of promoting S, L and/or M cone formation in a stem cell differentiation culture comprising supplementing the culture with:
(i) Triiodothyronine from about day 18 of cell differentiation;
(ii) DAPT from about day 28 to about day 42 of cell differentiation; and
(iii) retinoic acid for a first time period from about day 30 to about day 120 of cell differentiation.

21. The method according to claim 1, wherein said stem cell culture consists of human induced pluripotent stem cells (hiPSC) or human embryonic stem cells (hESC).

22. The method according to claim 1 further comprising isolating said synthetic retina from said cell culture medium.

23. The method of claim 22, further comprising a extracting at least one photoreceptor from said synthetic retina.

24. A synthetic retina obtainable by the method of claim 1.

25. A photoreceptor obtainable by the method of claim 1.

26. A pharmaceutical composition comprising said synthetic retina according to claim 24.

27. (canceled)

28. A method of treating a retinal disease or an ocular injury comprising implanting the synthetic retina according to claim 24 into the eye of a mammalian subject in need thereof.

29. The method according to claim 28, wherein the retinal disease is a retinal degenerative disease selected from the group consisting of Retinitis Pigmentosa, age-related macular degeneration, Bardet-Biedel syndrome, Bassen-Kornzweig syndrome, Best disease, choroideremia, gyrate atrophy, Leber congenital amaurosis, Refsun syndrome, Stargardt disease and Usher syndrome.

30. (canceled)

31. (canceled)

32. The method according to claim 28, wherein said stem cell culture consists of human induced pluripotent stem cells (hiPSC) obtained from a subject to be treated.

33. (canceled)

34. (canceled)
